(19) United States
(12) Patent Application Publication
(10) Pub. No.: US 2016/0030463 A1
(43) Pub. Date: Feb. 4, 2016

(54) COMPOSITIONS AND METHODS FOR TREATING OR PREVENTING INSULIN RESISTANCE OR ABNORMAL LEVELS OF CIRCULATING LIPIDS IN A MAMMAL

(71) Applicant: THE TRUSTEES OF THE UNIVERSITY OF PENNSYLVANIA, Philadelphia, PA (US)

(72) Inventor: Richard L Dunbar, Cherry Hill, NJ (US)

(21) Appl. No.: 14/773,903

(22) PCT Filed: Mar. 12, 2014
(86) PCT No.: PCT/US14/24742

§ 371 Date: Sep. 9, 2015

Related U.S. Application Data
(60) Provisional application No. 61/779,841, filed on Mar. 13, 2013.

(51) Int. Cl. A61K 31/714; A61K 31/519; A61K 31/675; A61K 31/455

(52) U.S. Cl. CPC A61K 31/714; A61K 31/455; A61K 31/519; A61K 31/675

(57) ABSTRACT

The present invention includes compositions useful for treating or preventing abnormal levels of circulating lipids in a mammal in need thereof, and methods using same. The compositions of the invention normalize levels of circulating lipids in the mammal, and do not cause the toxic side-effects known to occur with currently available lipid-managing medications, such as monotherapies using niacin or fibrates.

### CROSS-REFERENCE TO RELATED APPLICATIONS

The present application claims priority under 35 U.S.C. §119(e) to U.S. Provisional Application No. 61/779,841, filed Mar. 13, 2013, which application is hereby incorporated by reference in its entirety herein.

### STATEMENT REGARDING FEDERALLY SPONSORED RESEARCH OR DEVELOPMENT

This invention was made with government support under grant number K23HL091130 awarded by the National Heart, Lung, and Blood Institute (National Institutes of Health). The government has certain rights in the invention.

### BACKGROUND OF THE INVENTION

High total cholesterol (TC) levels, high triglyceride (TG) levels, low high-density lipoprotein (HDL) cholesterol levels, high low-density lipoprotein (LDL) cholesterol levels, and/or presence of small low-density lipoprotein particles have been associated with a variety of disease states, conditions and disorders in mammals.

Elevated serum cholesterol levels have been linked to coronary heart disease (CHD) (Badomin et al., 1992, Circulation 86(Supl. III):86-94). Cholesterol originates in the liver, where it is synthesized or isolated from dietary sources, and is transported in the circulatory system in the form of lipoproteins, which consist of complex aggregates of lipids and proteins responsible for lipid trafficking.

LDL and HDL are major lipoproteins in circulation. Cholesterol is transported from the liver to the tissues by LDL particles. Cholesterol is transported by HDL particles from the tissues to the liver, where cholesterol is catabolized and eliminated, in a process known as “reverse cholesterol transport.” HDL also removes non-cholesterol lipids, oxidized cholesterol and other oxidized products from the bloodstream.

Lipids may deposit in plaques within the arterial wall, giving rise to atherosclerotic plaques. The lipids in these plaques are mostly derived from apoprotein B-containing lipoproteins, such as LDL, IDL (intermediate-density lipoprotein) and VLDL (very low density lipoprotein). LDL is generally known as the “bad cholesterol.” On the other hand, HDL levels correlate inversely with coronary heart disease, and HDL is thought to be protective against coronary artery disease. Thus, HDL is generally known as “good cholesterol” and an atheroprotective lipoprotein.

In addition to HDL and LDL, triglycerides are also important markers of risk for cardiovascular disease. Numerous publications support the benefits of reducing fasting and postprandial triglyceride levels to avoid cardiovascular diseases, and therapeutic agents have been approved for this purpose. In recent years, experimental and clinical results have suggested that atherosclerosis is not simply a disease of lipid deposits. Rather, there is growing evidence that atherosclerosis has an inflammatory component that plays a critical role in the arterial plaque rupture that triggers most episodes of coronary thrombosis.

Dyslipidemia, sometimes referred to as lipoprotein abnormality, is a disorder generally defined by, among other factors, high cholesterol levels, high triglyceride levels, low HDL levels, high LDL levels, or presence of small low-density lipoprotein particles. Metabolic syndrome raises the risk for coronary heart disease, as a consequence of low HDL levels and high TG levels. This condition is defined as atherogenic dyslipidemia, as distinct from pure hypercholesterolemia (high LDL).

Nicotinic acid (niacin, also known as 3-pyridinecarboxylic acid or a salt thereof) administered in high doses is known to help correct dyslipidemia, primarily by reducing TG levels and elevating HDL levels (The National Cholesterol Education Program (NCEP) Expert Panel, “Third Report of the National Cholesterol Education Program (NCEP) Expert Panel on Detection, Evaluation, and Treatment of High Blood Cholesterol in Adults (Adult Treatment Panel III) Final Report,” Circulation 2002, 106(25):3143-421; The Coronary Drug Project Research Group, “Clofibrate and niacin in coronary heart disease,” JAMA 1975, 231(4):360-81; Canner et al., 2006, Am. J. Cardiol. 97(4):477-9). Nicotinic acid is also useful in improving atherogenic dyslipidemia.

However, nicotinic acid therapy causes unpleasant side effects, namely cutaneous vasodilation and intense flushing, as well as development of insulin resistance and drug-induced diabetes (Garg & Grundy, 1990, JAMA 264(6):723-6). An individual experiencing flushing may develop a visible, uncomfortable hot or flushed feeling upon administration of niacin. While certain materials and/or formulations have been suggested for avoiding or reducing cutaneous flushing (U.S. Pat. Nos. 4,956,252; 5,023,245; and 5,126,145), this unwanted side effect remains a problem for wide-scale therapeutic use of niacin products.

Fibrates are a class of amphipathic carboxylic acids used to treat hypercholesterolemia (high cholesterol). Fibrates commonly used comprise etofibrate, bezafibrate (Bezalip®), ciprofibrate (Modalim®), clofibrate, gemfibrozil (Lopid®), fenofibrate (TriCor®) and fenofibric acid (Trilipix®). Fibrates reduce the number of non-fatal heart attacks but do not improve all-cause mortality, and are therefore indicated only in those not tolerant to statins or those with significant triglyceridemia. Although less effective in lowering LDL levels, fibrates increase HDL levels and decrease TG levels. Most fibrates can cause mild stomach upset and myopathy (muscle pain with CPK elevations). Since fibrates increase the cholesterol content of bile, they increase the risk for gallstones. In combination with statin drugs, fibrates cause an increased risk of rhabdomyolysis (idiosyncratic destruction of muscle tissue), leading to renal failure.

There is a need in the art to identify compositions and methods that treat or prevent insulin resistance or abnormal levels of circulating lipids in a mammal. In one aspect, such compositions would be useful for treating or preventing dyslipidemia, wherein administration of the compositions reduces TG levels, reduces LDL levels, and/or reduces the prevalence of small, dense LDL, increases HDL levels in an individual, or a combination thereof. In another aspect, such compositions should have good efficacy in normalizing levels of circulating lipids in the mammal. In yet another aspect, such compositions should have good efficacy in treating or preventing insulin resistance in the mammal. Further, the compositions should not cause the toxic side-effects known to occur with currently available lipid-managing medications, such as monotherapies using niacin or fibrates. The present invention fulfills this need.

### BRIEF SUMMARY OF THE INVENTION

The present invention includes a method of reducing or preventing the increase of triglyceride levels in a mammal in need thereof. The method comprises administering to the mammal a therapeutically effective amount of a pharmaceutically acceptable composition comprising a lipid-managing medication. The mammal is further administered a methylation enhancing supplement. The lipid-managing medication comprises niacin, a fibrate, or a prodrug, analogue or metabolite thereof, thereby reducing or preventing the increase of triglyceride levels in the mammal.

The invention further includes a method of increasing or preventing the decrease of HDL levels in a mammal in need thereof. The method comprises administering to the mammal a therapeutically effective amount of a pharmaceutically acceptable composition comprising a lipid-managing medication. The mammal is further administered a methylation enhancing supplement wherein the lipid-managing medication comprises niacin, a fibrate, or a prodrug, analogue or metabolite thereof, thereby increasing or preventing the decrease of HDL levels in the mammal.

The invention also includes a pharmaceutical composition comprising a lipid-managing medication and a methylation enhancing supplement, wherein the lipid-managing medication comprises niacin or a fibrate, or a prodrug, analogue or metabolite thereof.

The invention further includes a method of reducing or preventing the increase of triglyceride levels in a mammal in need thereof. The method comprises administering to the mammal a therapeutically effective amount of a pharmaceutically acceptable composition comprising a methylation enhancing supplement, thereby reducing or preventing the increase of triglyceride levels in the mammal.

The invention also includes a method of increasing or preventing the decrease of HDL levels in a mammal in need thereof, the method comprising administering to the mammal a therapeutically effective amount of a pharmaceutically acceptable composition comprising a methylation enhancing supplement, thereby increasing or preventing the decrease of HDL levels in the mammal.

In certain embodiments, the methylation enhancing supplement comprises betaine, serine, methionine, s-adenosyl methionine, choline, phosphatidylcholine, creatine, cysteamine, cysteine, N-acetylcysteine, alpha lipoic acid, melatonin, silymarin, vitamin B5, pantethine, whey protein, vitamin B6, vitamin B9, vitamin B12, any salts thereof, or any combinations thereof, wherein the methylation enhancing supplement is not silymarin if the lipid-managing medication comprises niacin or a prodrug, analogue or metabolite thereof. In other embodiments, the lipid-managing medication and the methylation enhancing supplement are separately administered to the mammal. In yet other embodiments, the lipid-managing medication and the methylation enhancing supplement are concomitantly administered to the mammal. In yet other embodiments, the lipid-managing medication and the methylation enhancing supplement are co-formulated for administration to the mammal. In yet other embodiments, the lipid-managing medication is in a controlled release formulation. In yet other embodiments, lipid-managing medication is in an intermediate-release or extended-release formulation. In yet other embodiments, the mammal develops less insulin resistance than when the mammal is administered a similar dose of lipid-managing medication and is not administered the methylation enhancing supplement. In yet other embodiments, the mammal develops less tachyphylaxis than when the mammal is administered a similar dose of lipid-managing medication and is not administered the methylation enhancing supplement. In yet other embodiments, the mammal develops less hepatotoxicity than when the mammal is administered a similar dose of lipid-managing medication and is not administered the methylation enhancing supplement. In yet other embodiments, the mammal is administered from about 2 grams to about 6 grams of the lipid-managing medication daily. In yet other embodiments, the mammal is administered at least one daily dose selected from the group consisting of about 50 mg of vitamin B6, about 5 mg of vitamin B9, about 4 μg of vitamin B12, and about 2 grams to about 6 grams of betaine. In yet further embodiments, the mammal is a human.

### BRIEF DESCRIPTION OF THE DRAWINGS

For the purpose of illustrating the invention, there are depicted in the drawings certain embodiments of the invention. However, the invention is not limited to the precise arrangements and instrumentalities of the embodiments depicted in the drawings.

FIG. 1 is a Venn diagram illustrating homocysteinemia cases in mammals subjected to regimes of fasting homocysteine and/or standardized methionine load.

FIG. 2 is a schematic representation of niacin metabolism.

FIG. 3 is a schematic representation of methionine metabolism.

FIG. 4 is a non-limiting illustration of methylation enhancing supplements useful within the compositions and methods of the invention.

FIG. 5 is a schematic representation of a mechanism by which methyl depletion impairs VLDL synthesis.

FIG. 6 is a schematic representation of the net effect of methyl depletion on clinical lipid parameters.

FIG. 7 is a schematic illustration of downstream effects of homocysteinemia on HDL.

FIG. 8 is a schematic representation of effects of niacin on hepatocytes and adipocytes.

FIG. 9 is a schematic illustration of effects of homocysteine on hepatic insulin sensitivity.

FIG. 10 is a schematic illustration of effects of niacin on selected molecular markers.

FIG. 11 is a schematic illustration of effects of niacin on adipose insulin resistance.

FIG. 12 is a schematic illustration of a mechanism for niacin-induced insulin resistance.

FIG. 13 is a schematic illustration of a mechanism for niacin-induced hepatotoxicity.

FIG. 14 is a schematic illustration of effects of niacin on the inflammatory cascade.

FIG. 15 is a schematic illustration of an effect of niacin on platelet aggregation.

FIG. 16 is a fluxogram illustrating a non-limiting embodiment of the present study.

FIG. 17 is a table illustrating time and events overview for the optional extension study. Fasting lab panel includes: Complete blood count with differential. Chemistry Panel (Extended Electrolytes, BUN, creatine), Carbohydrate Panel (glucose, insulin, c-peptide, fructosamine. hemoglobin A1c), Hepatic Panel (ALT, AST, Bili, Albumin, Alk Phos, GCT, coagulation panel), methylation panel (homocysteine. SAM, SAH, vitamins B6, B9[folate], and B12) and lipids. ER niacin=SloNiacin®, IR niacin=Niacor®. Visit Windows: Starting with Visit 3, visits are scheduled at least 4 weeks apart. To accommodate subjects' schedules, a window of +2 weeks is allowable. Since a 4-week minimum is required between those visits, even when a visit is shifted within the 2-week window, subsequent visits will be shifted accordingly. Unscheduled visits are allowable ad hoc to follow up adverse events at the discretion of the PI. In that event, the latter unscheduled visits would not interfere with the timing of the scheduled visits discussed above.

FIG. 18 is a table summarizing the minimal whole blood requirements to cover fasting laboratory studies.

FIG. 19 is a table illustrating the study events for visits 2, 3 and 7, including determination of endogenous glucose production, the euglycemic hyperinsulinemic clamp, oral fat tolerance test, and methionine challenge.

FIG. 20 is a table illustrating the schedule and volumes for laboratory specimens collected.

FIG. 21 is a table summarizing the whole blood requirements for the study.

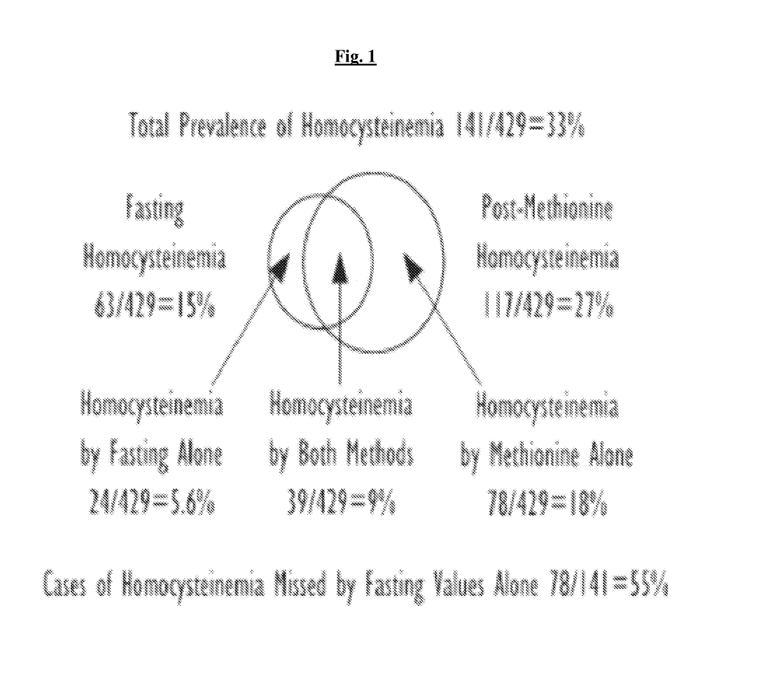

### DETAILED DESCRIPTION OF THE INVENTION

The present invention relates to the unexpected discovery that the administration to a mammal of a methylation enhancing supplement (i.e., a supplement that, for example, facilitates methyl transfer metabolism, decreases methylation burden or acts as a methyl group donor) along with a lipid-managing medication (such as niacin or a fibrate) prevents, moderates or treats the side effects of the lipid-managing medication and/or enhances the efficacy of the lipid-managing medication. In one embodiment, the lipid-managing medication and the supplement are administered simultaneously to the mammal. In another embodiment, the lipid-managing medication and the supplement are coformulated and coadministered to the mammal.

In one aspect, the combined administration of the lipid-managing medication and the methylation enhancing supplement to the mammal treats or prevents abnormal levels of circulating lipids in the mammal. In another aspect, the combined administration of the lipid-managing medication and the methylation enhancing supplement to the mammal treats or prevents insulin resistance in the mammal.

In one aspect, the administration of the methylation enhancing supplement, even in the absence of the lipid-managing medication, to the mammal treats or prevents abnormal levels of circulating lipids in the mammal. In another aspect, the administration of the methylation enhancing supplement, even in the absence of the lipid-managing medication, to the mammal treats or prevents insulin resistance in the mammal.

Non-limiting examples of supplements contemplated within the invention include betaine (trimethylglycine), serine, methionine, s-adenosyl methionine, choline, phosphatidylcholine, creatine, cysteamine, cysteine, N-acetylcysteine, silymarin, alpha lipoic acid, melatonin, vitamin B5 (pantothenic acid), pantethine, whey protein, vitamin B6, vitamin B9 and vitamin B12. In one embodiment, side effects of the lipid-managing medication comprise insulin resistance, hepatotoxicity or tachyphylaxis to the lipid-managing medication.

In one embodiment, the supplement prevents the elevation of, decreases, or moderates homocysteine levels in the mammal. In another embodiment, the combined administration of the supplement and the lipid-managing medication increases HDL or apolipoprotein A levels in the mammal. In yet another embodiment, the combined administration of the supplement and the lipid-managing medication decreases TG levels in the mammal.

The invention includes compositions comprising a methylation enhancing supplement and a lipid-managing medication. The invention further includes compounds comprising a methylation enhancing supplement, or derivative thereof, that is covalently bound to a lipid-managing medication, or derivative thereof.

### DEFINITIONS

As used herein, each of the following terms has the meaning associated with it in this section.

Unless defined otherwise, all technical and scientific terms used herein generally have the same meaning as commonly understood by one of ordinary skill in the art to which this invention belongs. Generally, the nomenclature used herein and the laboratory procedures in cell culture, molecular genetics, organic chemistry, and peptide chemistry are those well-known and commonly employed in the art.

As used herein, the articles “a” and “an” refer to one or to more than one (i.e., to at least one) of the grammatical object of the article. By way of example, “an element” means one element or more than one element.

As used herein, the term “about” is understood by persons of ordinary skill in the art and varies to some extent on the context in which it is used. “About” as used herein when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

As used herein, the term “methylation enhancing supplement” comprises a supplement that acts as methyl group donors in a mammal, facilitates methyl transfer metabolism in a mammal, or reduces methylation requirements in a mammal.

As used herein, the term “lipid-managing medication” refers to a therapeutic agent that, when administered to a subject, controls or manages one or more lipid levels in the subject, such as TC levels, LDL levels, HDL levels and/or TG levels, so that the levels of one or more lipids are modified to approach or reach levels routinely understood to be in the normal or non-health-threatening range for the subject.

As used herein, the term “tachyphylaxis” as relating to a medicament refers to any loss of efficacy of the medication seen over time, or attenuation of an adverse effect of the medication over time, or tolerance to the medication.

As used herein, the term “indocyanine green” refers to sodium 4-[2-[(1E,3E,5E,7Z)-7-[1,1-dimethyl-3-(4-sulfonatobutyl)benzo[e]indol-2-ylidene]hepta-1,3,5-trienyl]-1,1-dimethylbenzo[e]indol-3-ium-3-yl]butane-1-sulfonate, or any other cationic salt thereof.

As used herein, the term “AUC” refers to area under the curve.

As used herein, the term “C3” refers to complement C3.

As used herein, the term “CHD” refers to coronary heart disease.

As used herein, the term “CM” refers to chylomicron.

As used herein, the term “DI” refers to disposition index, derived from Bergman's Minimal Model applied to FSIGT data.

As used herein, the term “ΔAUC” refers to Delta AUC (comparing follow-up with baseline).

As used herein, the term “ER niacin” refers to extended-release niacin (such as Niaspan®, Nicolar® or Nialor®).

As used herein, the term “FCHL” refers to familial combined hyperlipoproteinemia.

As used herein, the term “FFA” refers to free fatty acid, also known as NEFA.

As used herein, the term “FSIGT” refers to frequently-sampled intravenous glucose tolerance test (for example, insulin-modified).

As used herein, the term “HDL” refers to high-density lipoprotein.

As used herein, the term “HSL” refers to hormone sensitive lipase.

As used herein, the term “HTG” refers to hypertriglyceridemia.

As used herein, the term “iAUC” refers to incremental area under the curve.

As used herein, the term “IR niacin” refers to immediate-release niacin (e.g., Niacor®).

As used herein, the term “LDL” refers to low-density lipoprotein.

As used herein, the term “LPL” refers to lipoprotein lipase.

As used herein, the term “MetSyn” refers to metabolic syndrome.

As used herein, the term “NA” refers to nicotinic acid (also known as niacin, a contraction of “nicotinic acid vitamin”).

As used herein, the term “NEFA” refers to non-esterified fatty acid, also known as FFA.

As used herein, the term “OFTT” refers to oral fat tolerance test.

As used herein, the term “Pbo” refers to placebo.

As used herein, the term “ppHDL” refers to postprandial HDL.

As used herein, the term “ppTG” refers to postprandial triglyceride.

As used herein, the term “RA” refers to retinoic acid.

As used herein, the term “SI” refers to insulin sensitivity index, derived from Bergman's Minimal Model applied to FSIGT data.

As used herein, the term “SD” refers to standard deviation.

As used herein, the term “sdLDL” refers to small, dense LDL.

As used herein, the term “SEM” refers to standard error of the mean.

As used herein, the term “T2DM” refers to Type 2 diabetes.

As used herein, the term “TG” refers to triglyceride/triacylglycerol.

As used herein, the term “VLDL” refers to very low-density lipoprotein.

As used herein, the terms “lipoprotein abnormality,” “dyslipidemia,” “hyperlipidemia,” and “hyperlipoproteinemia” are used interchangeably for the purpose of the present invention to describe unhealthy amounts of lipid or lipoprotein in the blood of a patient or subject. Such deviation may include one of the following occurrences, or any combination thereof: increase in cholesterol levels, increase in TG levels, decrease in HDL levels, increase in LDL levels, or increase in small-density lipoprotein particles.

As used herein, the term “antidyslipidemic agent” describes an agent that is designed to, when administered to a subject, treat the following conditions, prevent the onset of the following conditions, or any combination thereof, in the subject: high total cholesterol levels, high TG levels, low HDL levels, high LDL levels, or high small-density lipoprotein particle levels.

As used herein, the term “lipid profile” refers to a threshold-based assessment analysis of the lipid-related markers in the blood or plasma of a subject, wherein the thresholds for the lipid-markers are proposed by the National Cholesterol Education program (NCEP) of the National Institutes of Health (NIH). The threshold values currently in use are based on the Third Report of the Expert Panel on Detection, Evaluation, and Treatment of High Blood Cholesterol in Adults (Adult Treatment Panel III, or ATP III), and may be easily adapted to any upcoming revision of these guidelines. The present invention encompasses and adopts any future updates of the threshold values. The lipid profile indicates how each lipid marker of the subject compares with healthy or unhealthy ranges of the lipid markers defined by doctors or other medical specialists, and facilitates development of a therapeutic intervention to improve the levels of one or more lipid markers of the subject.

As used herein, the term “bioavailability” denotes the degree to which a drug or other substance becomes available to the target tissue and/or systemic circulation after administration.

As used herein, the term “treatment” or “treating” is defined as the application or administration to a subject of a therapeutic agent, i.e., a compound useful within the invention (alone or in combination with another pharmaceutical agent), or application or administration of a therapeutic agent to an isolated tissue or cell line from a subject (e.g., for diagnosis or ex vivo applications), who has dyslipidemia, a symptom of dyslipidemia or the potential to develop dyslipidemia, with the purpose to cure, heal, alleviate, relieve, alter, remedy, ameliorate, improve or affect dyslipidemia, the symptoms of dyslipidemia or the potential to develop dyslipidemia. Such treatments may be specifically tailored or modified, based on knowledge obtained from the field of pharmacogenomics.

As used herein, the term “insulin resistance” refers to all forms of disrupted insulin or glucose regulation, including diminished insulin sensitivity (which itself may be comprised of diminished insulin-mediated suppression of endogenous glucose production or diminished insulin-mediated glucose uptake), diminished glucose sensitivity, also called glucose effectiveness (which itself may be comprised of diminished glucose-mediated suppression of endogenous glucose production or diminished glucose-mediated suppression of glucose uptake), increased endogenous glucose production (which itself may be comprised of increased gluconeogenesis or increased glycogenolysis), diminished insulin secretion, or altered insulin catabolism. Likewise, as used herein, the term “insulin sensitivity” is used as a metonym for normal glucose regulation including the several aspects listed above under “insulin resistance.”

As used herein, the term “prevent” or “prevention” means no disorder or disease development if none had occurred, or no further disorder or disease development if there had already been development of the disorder or disease. Also considered is the ability of one to prevent some or all of the symptoms associated with the disorder or disease.

As used herein, the term “patient” or “subject” or “individual” refers to a mammal, wherein the mammal may be human or non-human. These terms may be used interchangeably within the disclosure of the invention. Non-human animals include, for example, livestock or pets, such as ovine, bovine, porcine, canine, feline and murine mammals. Preferably, the patient or subject is human.

As used herein, the terms “effective amount,” “pharmaceutically effective amount,” and “therapeutically effective amount” refer to a non-toxic but sufficient amount of an agent to provide the desired biological result. That result can be reduction and/or alleviation of the signs, symptoms, or causes of a disease, or any other desired alteration of a biological system. An appropriate therapeutic amount in any individual case may be determined by one of ordinary skill in the art using routine experimentation.

As used herein, the term “pharmaceutically acceptable” refers to a material, such as a carrier or diluent, which does not abrogate the biological activity or properties of the compound, and is relatively non-toxic, i.e., the material may be administered to an individual without causing undesirable biological effects or interacting in a deleterious manner with any of the components of the composition in which it is contained.

As used herein, the term “pharmaceutical composition” refers to a mixture of at least one compound useful within the invention with other chemical components, such as carriers, stabilizers, diluents, dispersing agents, suspending agents, thickening agents, and/or excipients. The pharmaceutical composition facilitates administration of the compound to an organism. Multiple techniques of administering a compound exist in the art including, but not limited to: intravenous, oral, aerosol, parenteral, ophthalmic, pulmonary and topical administration.

The term “pharmaceutically acceptable carrier” includes a pharmaceutically acceptable salt, pharmaceutically acceptable material, composition or carrier, such as a liquid or solid filler, diluent, excipient, solvent or encapsulating material, involved in carrying or transporting a compound(s) of the present invention within or to the subject such that it may perform its intended function. Typically, such compounds are carried or transported from one organ, or portion of the body, to another organ, or portion of the body. Each salt or carrier must be “acceptable” in the sense of being compatible with the other ingredients of the formulation, and not injurious to the subject. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; diluent; granulating agent; lubricant; binder; disintegrating agent; wetting agent; emulsifier; coloring agent; release agent; coating agent; sweetening agent; flavoring agent; perfuming agent; preservative; antioxidant; plasticizer; gelling agent; thickener; hardener; setting agent; suspending agent; surfactant; humectant; carrier; stabilizer; and other non-toxic compatible substances employed in pharmaceutical formulations, or any combination thereof.

As used herein, “pharmaceutically acceptable carrier” also includes any and all coatings, antibacterial and antifungal agents, and absorption delaying agents, and the like that are compatible with the activity of the compound, and are physiologically acceptable to the subject. Supplementary active compounds may also be incorporated into the compositions. The carrier may be a solvent or dispersion medium containing, for example, water, ethanol, polyol (for example, glycerol, propylene glycol, and liquid polyethylene glycol, and the like), suitable mixtures thereof, and vegetable oils. The proper fluidity may be maintained, for example, by the use of a coating such as lecithin, by the maintenance of the required particle size in the case of dispersion and by the use of surfactants. Prevention of the action of microorganisms may be achieved by various antibacterial and antifungal agents, for example, parabens, chlorobutanol, phenol, ascorbic acid, thimerosal, and the like. In many cases, it is preferable to include isotonic agents, for example, sugars, sodium chloride, or polyalcohols such as mannitol and sorbitol, in the composition. Prolonged absorption of the injectable compositions may be brought about by including in the composition an agent which delays absorption, for example, aluminum monostearate or gelatin.

As used herein, the term “salt” embraces addition salts of free acids or free bases that are compounds useful within the invention.

As used herein, the term “pharmaceutically acceptable salt” refers to a salt of the administered compounds prepared from pharmaceutically acceptable non-toxic acids, including inorganic acids, organic acids, inorganic bases, organic bases, solvates, hydrates, or clathrates thereof. Suitable acid addition salts may be prepared from an inorganic acid or from an organic acid. Examples of inorganic acids include hydrochloric, hydrobromic, hydriodic, nitric, carbonic, sulfuric, phosphoric acids, perchloric and tetrafluoroboronic acids. Appropriate organic acids may be selected from aliphatic, cycloaliphatic, aromatic, araliphatic, heterocyclic, carboxylic and sulfonic classes of organic acids, examples of which include formic, acetic, propionic, succinic, glycolic, gluconic, lactic, malic, tartaric, citric, ascorbic, glucuronic, maleic, fumaric, pyruvic, aspartic, glutamic, benzoic, anthranilic, 4-hydroxybenzoic, phenylacetic, mandelic, embonic (pamoic), methanesulfonic, ethanesulfonic, benzenesulfonic, pantothenic, trifluoromethanesulfonic, 2-hydroxyethanesulfonic, p-toluenesulfonic, sulfanilic, cyclohexylaminosulfonic, stearic, alginic, β-hydroxybutyric, salicylic, galactaric and galacturonic acid. Suitable base addition salts of compounds useful within the invention include, for example, metallic salts including alkali metal, alkaline earth metal and transition metal salts such as, for example, lithium, calcium, magnesium, potassium, ammonium, sodium and zinc salts. Acceptable base addition salts also include organic salts made from basic amines such as, for example, N,N′-dibenzylethylenediamine, chloroprocaine, choline, diethanolamine, ethylenediamine, meglumine (N-methyl-glucamine) and procaine. All of these salts may be prepared by conventional means from the corresponding free base compound by reacting, for example, the appropriate acid or base with the corresponding free base.

As used herein, the term “instructional material” includes a publication, a recording, a diagram, or any other medium of expression that may be used to communicate the usefulness of the compounds useful within the invention. In some instances, the instructional material may be part of a kit useful for effecting alleviating or treating the various diseases or disorders recited herein. Optionally, or alternately, the instructional material may describe one or more methods of alleviating the diseases or disorders in a cell or a tissue of a mammal. The instructional material of the kit may, for example, be affixed to a container that contains the compounds useful within the invention or be shipped together with a container that contains the compounds. Alternatively, the instructional material may be shipped separately from the container with the intention that the recipient uses the instructional material and the compound cooperatively. For example, the instructional material is for use of a kit; instructions for use of the compound; or instructions for use of a formulation of the compound.

As used herein, the term “alkyl,” by itself or as part of another substituent means, unless otherwise stated, a straight or branched chain hydrocarbon having the number of carbon atoms designated (i.e. C₁₋₆ means one to six carbon atoms) and includes straight, branched chain, or cyclic substituent groups. Examples include methyl, ethyl, propyl, isopropyl, butyl, isobutyl, tert-butyl, pentyl, neopentyl, hexyl, and cyclopropylmethyl. Most preferred is (C₁-C₆)alkyl, particularly ethyl, methyl, isopropyl, isobutyl, n-pentyl, n-hexyl and cyclopropylmethyl.

As used herein, the term “substituted alkyl” means alkyl as defined above, substituted by one, two or three substituents selected from the group consisting of halogen, —OH, alkoxy, —NH₂, —N(CH₃)₂, —C(═O)OH, —CF₃, —C≡N (or —CN), —C(═O)O(C₁-C₄)alkyl, —C(═O)NH₂, —SO₂NH₂, —C(═NH)NH₂, and —NO₂, preferably containing one or two substituents selected from halogen, —OH, alkoxy, —NH₂, —CF₃, —N(CH₃)₂, and —C(═O)OH, more preferably selected from halogen, alkoxy and —OH. Examples of substituted alkyls include, but are not limited to, 2,2-difluoropropyl, 2-carboxycyclopentyl and 3-chloropropyl.

As used herein, the term “heteroalkyl” by itself or in combination with another term means, unless otherwise stated, a stable straight or branched chain alkyl group consisting of the stated number of carbon atoms and one or two heteroatoms selected from the group consisting of O, N, and S, and wherein the nitrogen and sulfur atoms may be optionally oxidized and the nitrogen heteroatom may be optionally quaternized. The heteroatom(s) may be placed at any position of the heteroalkyl group, including between the rest of the heteroalkyl group and the fragment to which it is attached, as well as attached to the most distal carbon atom in the heteroalkyl group. Examples include: —O—CH₂—CH₂—CH₃, —CH₂—CH₂—CH₂—OH, —CH₂—CH₂—NH—CH₃, —CH₂—S—CH₂—CH₃, and —CH₂CH₂—S(═O)—CH₃. Up to two heteroatoms may be consecutive, such as, for example, —CH₂—NH—OCH₃, or —CH₂—CH₂—S—S—CH₃

As used herein, the term “alkoxy” employed alone or in combination with other terms means, unless otherwise stated, an alkyl group having the designated number of carbon atoms, as defined above, connected to the rest of the molecule via an oxygen atom, such as, for example, methoxy, ethoxy, 1-propoxy, 2-propoxy(isopropoxy) and the higher homologs and isomers. Preferred are (C₁-C₃) alkoxy, particularly ethoxy and methoxy.

As used herein, the term “halo” or “halogen” alone or as part of another substituent means, unless otherwise stated, a fluorine, chlorine, bromine, or iodine atom, preferably, fluorine, chlorine, or bromine, more preferably, fluorine or chlorine.

As used herein, the term “cycloalkyl” refers to a mono cyclic or polycyclic non-aromatic radical, wherein each of the atoms forming the ring (i.e. skeletal atoms) is a carbon atom. In one embodiment, the cycloalkyl group is saturated or partially unsaturated. In another embodiment, the cycloalkyl group is fused with an aromatic ring. Cycloalkyl groups include groups having from 3 to 10 ring atoms. Illustrative examples of cycloalkyl groups include, but are not limited to, the following moieties:

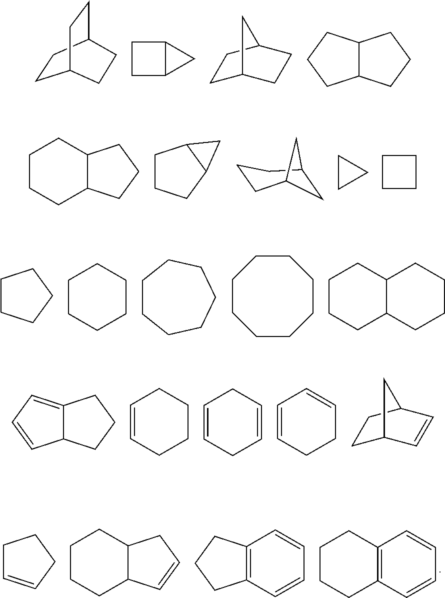

Monocyclic cycloalkyls include, but are not limited to, cyclopropyl, cyclobutyl, cyclopentyl, cyclohexyl, cycloheptyl, and cyclooctyl. Dicyclic cycloalkyls include, but are not limited to, tetrahydronaphthyl, indanyl, and tetrahydropentalene. Polycyclic cycloalkyls include adamantine and norbornane. The term cycloalkyl includes “unsaturated nonaromatic carbocyclyl” or “nonaromatic unsaturated carbocyclyl” groups, both of which refer to a nonaromatic carbocycle as defined herein, which contains at least one carbon carbon double bond or one carbon carbon triple bond.

As used herein, the term “heterocycloalkyl” or “heterocyclyl” refers to a heteroalicyclic group containing one to four ring heteroatoms each selected from O, S and N. In one embodiment, each heterocycloalkyl group has from 4 to 10 atoms in its ring system, with the proviso that the ring of said group does not contain two adjacent O or S atoms. In another embodiment, the heterocycloalkyl group is fused with an aromatic ring. In one embodiment, the nitrogen and sulfur heteroatoms may be optionally oxidized, and the nitrogen atom may be optionally quaternized. The heterocyclic system may be attached, unless otherwise stated, at any heteroatom or carbon atom that affords a stable structure. A heterocycle may be aromatic or non-aromatic in nature. In one embodiment, the heterocycle is a heteroaryl.

An example of a 3-membered heterocycloalkyl group includes, and is not limited to, aziridine. Examples of 4-membered heterocycloalkyl groups include, and are not limited to, azetidine and a beta lactam. Examples of 5-membered heterocycloalkyl groups include, and are not limited to, pyrrolidine, oxazolidine and thiazolidinedione. Examples of 6-membered heterocycloalkyl groups include, and are not limited to, piperidine, morpholine and piperazine. Other non-limiting examples of heterocycloalkyl groups are:

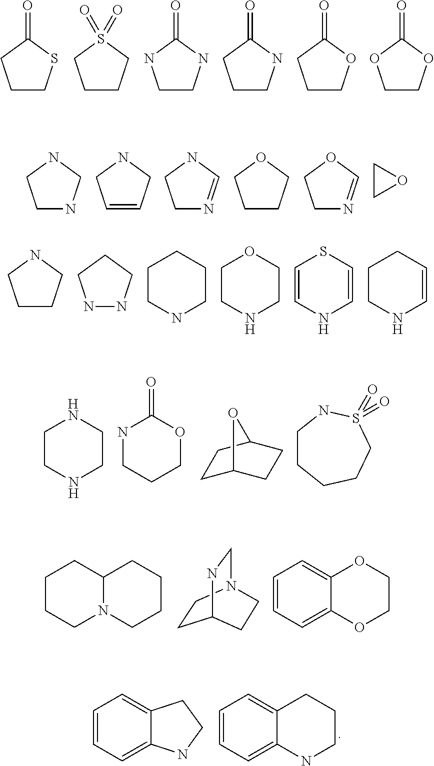

Examples of non-aromatic heterocycles include monocyclic groups such as aziridine, oxirane, thiirane, azetidine, oxetane, thietane, pyrrolidine, pyrroline, pyrazolidine, imidazoline, dioxolane, sulfolane, 2,3-dihydrofuran, 2,5-dihydrofuran, tetrahydrofuran, thiophane, piperidine, 1,2,3,6-tetrahydropyridine, 1,4-dihydropyridine, piperazine, morpholine, thiomorpholine, pyran, 2,3-dihydropyran, tetrahydropyran, 1,4-dioxane, 1,3-dioxane, homopiperazine, homopiperidine, 1,3-dioxepane, 4,7-dihydro-1,3-dioxepin, and hexamethyleneoxide.

As used herein, the term “aromatic” refers to a carbocycle or heterocycle with one or more polyunsaturated rings and having aromatic character, i.e., having (4n+2) delocalized π (pi) electrons, where n is an integer.

As used herein, the term “aryl,” employed alone or in combination with other terms, means, unless otherwise stated, a carbocyclic aromatic system containing one or more rings (typically one, two or three rings), wherein such rings may be attached together in a pendent manner, such as a biphenyl, or may be fused, such as naphthalene. Examples of aryl groups include phenyl, anthracyl, and naphthyl. Preferred examples are phenyl and naphthyl, most preferred is phenyl.

As used herein, the term “aryl-(C₁-C₃)alkyl” means a functional group wherein a one- to three-carbon alkylene chain is attached to an aryl group, e.g., —CH₂CH₂-phenyl. Preferred is aryl-CH₂— and aryl-CH(CH₃)—. The term “substituted aryl-(C₁-C₃)alkyl” means an aryl-(C₁-C₃)alkyl functional group in which the aryl group is substituted. Preferred is substituted aryl(CH₂)—. Similarly, the term “heteroaryl-(C₁-C₃)alkyl” means a functional group wherein a one to three carbon alkylene chain is attached to a heteroaryl group, e.g., —CH₂CH₂-pyridyl. Preferred is heteroaryl-(CH₂)—. The term “substituted heteroaryl-(C₁-C₃)alkyl” means a heteroaryl-(C₁-C₃)alkyl functional group in which the heteroaryl group is substituted. Preferred is substituted heteroaryl-(CH₂)—.

As used herein, the term “heteroaryl” or “heteroaromatic” refers to a heterocycle having aromatic character. A polycyclic heteroaryl may include one or more rings that are partially saturated. Examples include the following moieties:

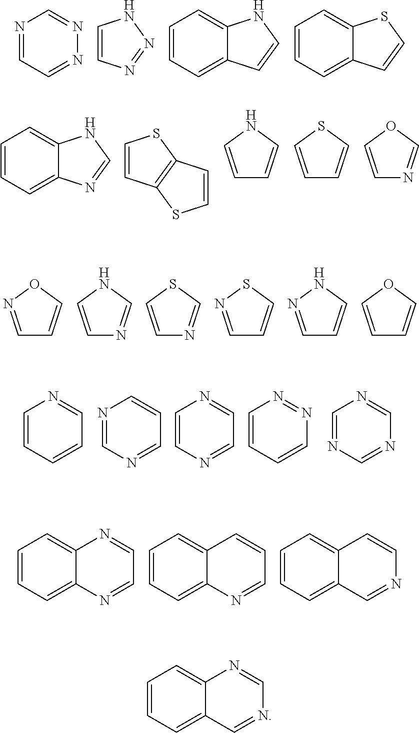

Examples of heteroaryl groups also include pyridyl, pyrazinyl, pyrimidinyl (particularly 2- and 4-pyrimidinyl), pyridazinyl, thienyl, furyl, pyrrolyl (particularly 2-pyrrolyl), imidazolyl, thiazolyl, oxazolyl, pyrazolyl (particularly 3- and 5-pyrazolyl), isothiazolyl, 1,2,3-triazolyl, 1,2,4-triazolyl, 1,3,4-triazolyl, tetrazolyl, 1,2,3-thiadiazolyl, 1,2,3-oxadiazolyl, 1,3,4-thiadiazolyl and 1,3,4-oxadiazolyl.

Examples of polycyclic heterocycles and heteroaryls include indolyl (particularly 3-, 4-, 5-, 6- and 7-indolyl), indolinyl, quinolyl, tetrahydroquinolyl, isoquinolyl (particularly 1- and 5-isoquinolyl), 1,2,3,4-tetrahydroisoquinolyl, cinnolinyl, quinoxalinyl (particularly 2- and 5-quinoxalinyl), quinazolinyl, phthalazinyl, 1,8-naphthyridinyl, 1,4-benzodioxanyl, coumarin, dihydrocoumarin, 1,5-naphthyridinyl, benzofuryl (particularly 3-, 4-, 5-, 6- and 7-benzofuryl), 2,3-dihydrobenzofuryl, 1,2-benzisoxazolyl, benzothienyl (particularly 3-, 4-, 5-, 6-, and 7-benzothienyl), benzoxazolyl, benzothiazolyl (particularly 2-benzothiazolyl and 5-benzothiazolyl), purinyl, benzimidazolyl (particularly 2-benzimidazolyl), benzotriazolyl, thioxanthinyl, carbazolyl, carbolinyl, acridinyl, pyrrolizidinyl, and quinolizidinyl.

As used herein, the term “substituted” means that an atom or group of atoms has replaced hydrogen as the substituent attached to another group. The term “substituted” further refers to any level of substitution, namely mono-, di-, tri-, tetra-, or penta-substitution, where such substitution is permitted. The substituents are independently selected, and substitution may be at any chemically accessible position. In one embodiment, the substituents vary in number between one and four. In another embodiment, the substituents vary in number between one and three. In yet another embodiment, the substituents vary in number between one and two.

As used herein, the term “optionally substituted” means that the referenced group may be substituted or unsubstituted. In one embodiment, the referenced group is optionally substituted with zero substituents, i.e., the referenced group is unsubstituted. In another embodiment, the referenced group is optionally substituted with one or more additional group(s) individually and independently selected from groups described herein.

In one embodiment, the substituents are independently selected from the group consisting of oxo, halogen, —CN, —NH₂, —OH, —NH(CH₃), —N(CH₃)₂, alkyl (including straight chain, branched and/or unsaturated alkyl), substituted or unsubstituted cycloalkyl, substituted or unsubstituted heterocycloalkyl, fluoro alkyl, substituted or unsubstituted heteroalkyl, substituted or unsubstituted alkoxy, fluoroalkoxy, —S-alkyl, S(═O)₂alkyl, —C(═O)NH[substituted or unsubstituted alkyl, or substituted or unsubstituted phenyl], —C(═O)N[H or alkyl]₂, —OC(═O)N[substituted or unsubstituted alkyl]₂, —NHC(═O)NH[substituted or unsubstituted alkyl, or substituted or unsubstituted phenyl], —NHC(═O)alkyl, —N[substituted or unsubstituted alkyl]C(═O)[substituted or unsubstituted alkyl], —NHC(═O)[substituted or unsubstituted alkyl], —C(OH)[substituted or unsubstituted alkyl]₂, and —C(NH₂)[substituted or unsubstituted alkyl]₂. In another embodiment, by way of example, an optional substituent is selected from oxo, fluorine, chlorine, bromine, iodine, —CN, —NH₂, —OH, —NH(CH₃), —N(CH₃)₂, —CH₃, —CH₂CH₃, —CH(CH₃)₂, —CF₃, —CH₂CF₃, —OCH₃, —OCH₂CH₃, —OCH(CH₃)₂, —OCF₃, —OCH₂CF₃, —S(═O)₂—CH₃, —C(═O)NH₂, —C(═O)—NHCH₃, —NHC(═O)NHCH₃, —C(═O)CH₃, and —C(═O)OH. In yet one embodiment, the substituents are independently selected from the group consisting of C₁₋₆ alkyl, —OH, C₁₋₆ alkoxy, halo, amino, acetamido, oxo and nitro. In yet another embodiment, the substituents are independently selected from the group consisting of C₁₋₆ alkyl, C₁₋₆ alkoxy, halo, acetamido, and nitro. As used herein, where a substituent is an alkyl or alkoxy group, the carbon chain may be branched, straight or cyclic, with straight being preferred.

### DESCRIPTION

Niacin and fibrates are indicated to improve dyslipidemia and raise HDL levels in a mammal However, administration of either class of drugs is generally accompanied by undesirable side effects in the mammal, such as increased homocysteine levels over the patient's pre-treatment baseline and/or over the upper limit of normal (i.e., relative or absolute homocysteinemia).

Prodrugs, analogues or metabolites of niacin or fibrates, which may be used to improve dyslipidemia in a mammal, may also raise homocysteine when administered to the mammal. Non-limiting examples of niacin prodrugs contemplated within the invention include nicotinyl alcohol and niacin bound to other chemical entities (e.g., inositol hexanicotinate/myo inositol hexanicotinate [Hexopal®], sorbitol hexanicotinate [sorbinicate], niceritrol [pentaerythritol-tetranicotinate], nicofuranose [Bradilan®, vasperdil], etofibrate, niacin-bound chromium). Non-limiting examples of niacin metabolites contemplated within the invention include nicotinuric acid, nicotinic acid N-methylbetaine (trigonelline), 6-hydroxynicotinic acid, nicotinate mononucleotide, nicotinate adenine dinucleotide, nicotinamide adenine dinucleotide, nicotinamide, nicotinamide-N-oxide, N-methylnicotinamide, N-methyl-2-pyridone, N-methyl-4-pyridone-5-carboxamide and 6-hydroxynicotinamide. Non-limiting examples of niacin analogues contemplated within the invention include acipimox and acifran. Non-limiting examples of fibrates contemplated within the invention include clofibrate, gemfibrozil, etofibrate, fenofibrate, fenofibric acid, bezofibrate and ciprofibrate.

In one embodiment, niacin as contemplated or used within the compositions and methods of the invention is interchangeable with a prodrug, modification, or metabolite of niacin.

In one embodiment, a fibrate as contemplated used within the compositions and methods of the invention is interchangeable with a prodrug, modification, or metabolite of a fibrate.

Several supplements are known to act as methyl group donors, facilitate methyl transfer metabolism, or otherwise reduce methylation requirements (for example, by providing the product of a methylation reaction and thereby reducing demand for methyl groups; for example, creatine). These supplements are hereafter referred to as “methylation enhancing supplements.”

Non-limiting examples of methylation enhancing supplements that provide methyl groups include, but are not limited to, betaine (trimethylglycine), serine, methionine, s-adenosyl methionine, choline, and phosphatidylcholine.

Non-limiting examples of methylation enhancing supplements that decrease methylation burden include, but are not limited to, creatine, cysteamine, cysteine, N-acetylcysteine, silymarin or any component thereof, alpha lipoic acid, melatonin, vitamin B5 (pantothenic acid), pantethine and whey protein. In one embodiment, the supplement is not silymarin if the lipid-managing medication comprises niacin or a prodrug, analogue or metabolite thereof.

Non-limiting examples of methylation enhancing supplements that facilitate methyl transfer include, but are not limited to, vitamin B6, vitamin B9, and vitamin B12.

Non-limiting examples of pathways in homocysteinemia are shown in Table 1, along with possible therapeutic treatments.

TABLE 1

| Pathway Affected | | | Possible Treatment |
|---|---|---|---|
| A. | Insufficient Homocysteine Recycling | | |
| | 1. | Endogenous methyl pool depletion | Methyl Donors: |
| | | | Supplemental betaine |
| | | | Supplemental serine |
| | 2. | Relative-functional deficiency of | Methyl-Transfer Agents: |
| | | methyl-transferring vitamins that | Supplemental B6/B9/B12 |
| | | mediate recycling (B6/B9/B12) | |
| | 3. | Combination of methyl pool | Either or both of the |
| | | depletion and deficiency of | strategies above |
| | | methyl-transferring vitamins | |

| | | |
|---|---|---|
| B. | Insufficient Homocysteine | Methyl Donor and Transfer |
| | Transulfuration | Agent: Supplemental serine |
| | | Supplemental B6 |

Metanx® is a combination of pyridoxal 5′-phosphate (the active form of vitamin B6), L-methylfolate calcium (Metafolin®, the active stereoisomer of folic acid, also known as vitamin B9), and methylcobalamin (the active form of vitamin B12). The FDA approved Metanx® as a medicinal food available by prescription only. In one embodiment, a capsule of Metanx® comprises 35 mg pyridoxal 5′-phosphate, 3 mg L-methylfolate calcium, and 2 mg methylcobalamin. The typical dose in humans is 2 capsules daily.

Foltx® is a combination of the vitamin prodrugs: folacin (folic acid); pyridoxine (vitamin B6) and cyanocobalamin (vitamin B12). In one embodiment, a single dose of Foltx® comprises 2.5 mg folacin, 25 mg pyridoxine and 2 mg cyanocobalamin. The typical dose in humans is 2 capsules daily.

CerefolinNAC® is a combination of L-methylfolate calcium (Metafolin®, the active stereoisomer of folic acid, also known as vitamin B9), methylcobalamin (the active form of vitamin B12), and n-acetylcysteine. In one embodiment, a single dose of CerefolinNAC® comprises 6 mg L-methylfolate calcium, 2 mg methylcobalamin, and 600 mg n-acetylcysteine. The typical dose in humans is 1 capsule daily.

The side effects of the lipid-managing medication comprise insulin resistance, hepatotoxicity or tachyphylaxis (i.e., loss of efficacy) to the lipid-managing medication. As described herein, co-administration to the mammal of methylation-enhancing supplements along with niacin or a fibrate opposes the elevation of homocysteine levels that result from the administration of the niacin or fibrate. In one embodiment, administration of methylation-enhancing supplements results in increased HDL or apolipoprotein A levels in the mammal, thus augmenting the benefit of niacin or fibrates. In another embodiment, co-administration of methylation-enhancing supplements to a mammal receiving niacin therapy opposes niacin-induced insulin resistance or augments favorable effects of fibrates on insulin sensitivity. In yet another embodiment, co-administration of methylation-enhancing supplements to a mammal receiving niacin or fibrate therapy opposes niacin-induced or fibrate-induced hepatotoxicity.

Although niacin and fibrates increase HDL levels and reduces TG levels in a mammal, niacin in particular induces insulin resistance, glucose resistance, glucose intolerance, hyperglycemia and drug-induced diabetes in the mammal. Further, tachyphylaxis has also been observed in relation to the HDL-raising effect of niacin and fibrates. For example, the final increments in HDL and apolipoprotein A-I levels were only half of the initial benefit provided by niacin, and similar diminutions have been reported with fibrates. Administration of niacin or a fibrate to a mammal induces relative or absolute homocysteinemia, which inhibits apo A-I production and thus decreases HDL levels. This explains the tachyphylaxis effects of niacin and other homocysteine-raising, lipid-altering drugs such as fibrates. The homocysteinemia caused by administration of niacin also decreases insulin sensitivity and thus potentiates insulin resistance in the mammal. Likewise, the homocysteinemia caused by administration of a fibrate may cause decrease insulin sensitivity and reverse favorable effects of the fibrate itself, or even promote net insulin resistance, by offsetting potential insulin-sensitizing effects of fibrates.

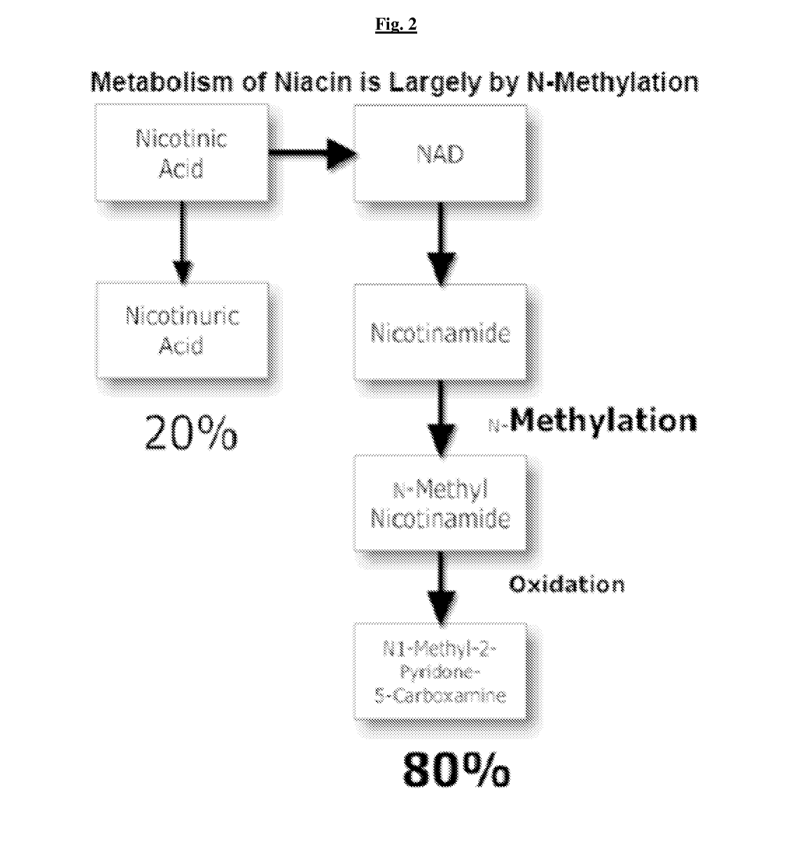

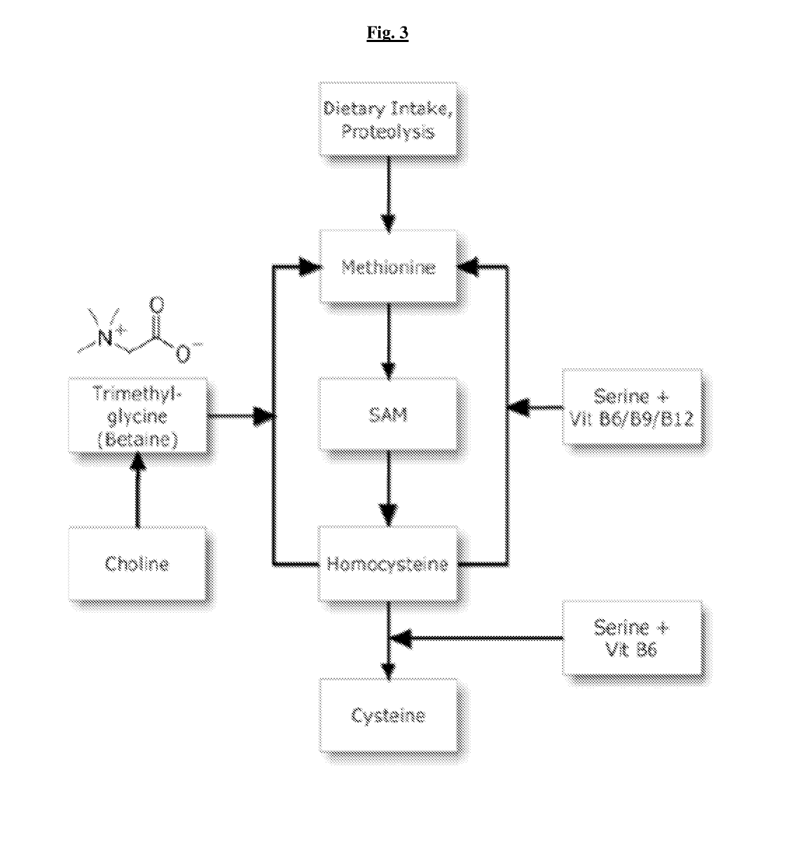

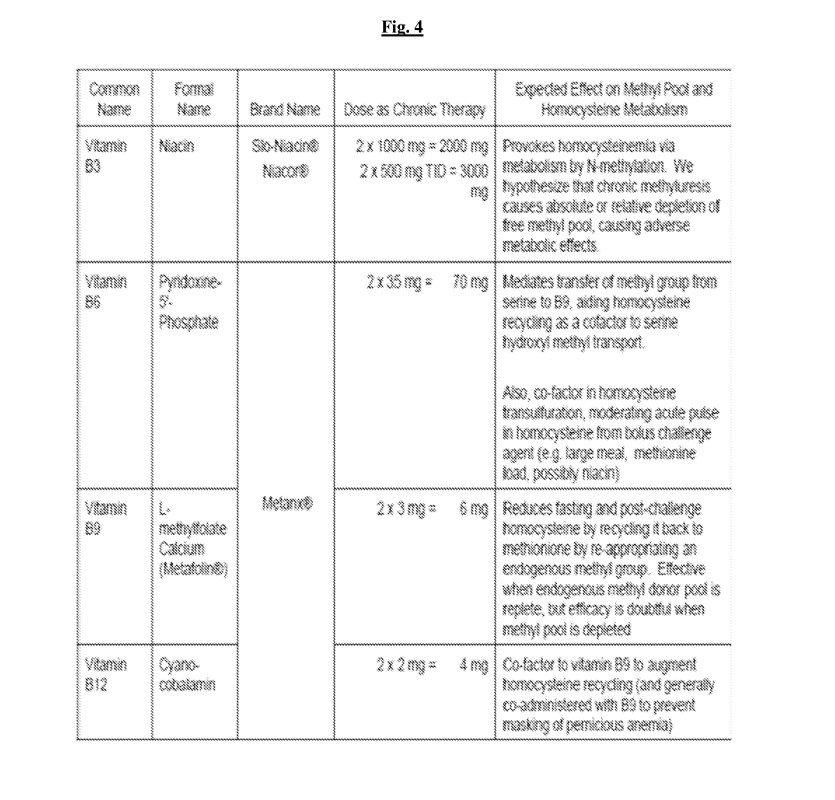

Increased homocysteine levels result from aggressive utilization of methyl groups during metabolism. For example, relative or absolute niacin-induced homocysteinemia may be triggered by relative methyl depletion (by consumption of methyl reserves to form N-methyl-nicotinamide; FIG. 2, and to a lesser extent, methylation of nicotinic acid to N-methyl nicotinate, i.e., trigonelline), by methyl uresis (wherein methylated derivatives of niacin are eliminated in urine) or by vitamin deficiency (wherein methyl-donating vitamin co-factors are consumed). Chronic overutilization of methyl reserves is also responsible for other side effects, including niacin-induced insulin resistance, tachyphylaxis to efficacy, and hepatotoxicity. Daily mega-doses of niacin may cause sustained methyl uresis, and this chronic elimination of methyl groups eventually impairs homocysteine recycling by methyl group substrate limitation. Chronic increases in methionine-homocysteine flux erode the capacity of vitamin cofactors to meet the increased demand. In a clinical setting, relative or absolute homocysteinemia may be more easily identified by methionine challenge (post-methionine loading), rather than fasting homocysteine alone. In fact, the majority of mammals would escape homocysteinemia detection under fasting homocysteine conditions alone (van der Griend et al., 1998, J. Lab. Clin. Med. 132(1):67).

There is a complex balance between the niacin's therapeutic effects and its side effects. Niacin may limit methylation capacity and thereby limit hepatic production of phosphatidyl choline. Since the latter is an essential substrate for VLDL synthesis, this implicates disrupted methylation metabolism as a possible cause of the drug's TG- and LDL-lowering efficacy, because TGs and cholesterol are “trapped” in the liver. If this mechanism underlies some of niacin's efficacy, trapping TGs in the liver may predispose patients to fatty liver and hepatotoxicity, both of which are reported in the niacin literature. Disrupted methylation metabolism also applies to administration of fibrates to a mammal.

The invention includes the administration of methylation-enhancing supplements to a mammal undergoing niacin or fibrate treatment, as a means of mitigating the side effects of either compound. Supplements that, in a non-limiting example, enhance methylation metabolism, serve as methyl donors or reduce demand for methyl groups are contemplated within the invention. Such methylation-enhancing supplements comprise, but are not limited to, betaine (trimethylglycine), N-methylnicotinate (trigonelline), vitamin B5, pantethine, whey protein, vitamin B6 (including all moieties thereof), vitamin B9 (including all moieties thereof, including folate), vitamin B12 (including all moieties thereof), cysteamine, cysteine, N-acetylcysteine, silymarin and any components thereof, alpha lipoic acid, melatonin, serine, methionine, s-adenosyl methionine, creatine, glutathione, phosphatidylserine, phosphatidylcholine, phosphatidylethanolamine, and choline. The invention also includes the administration of methylation-enhancing supplements to a mammal undergoing treatment with a prodrug, analogue or metabolite of niacin or a fibrate, as a means of mitigating the side effects of either compound. Any of these supplements, or any combination thereof, may be used to offset homocysteinemia resulting from treatment with niacin or a fibrate (or a prodrug, analogue or metabolite thereof), and serve to potentiate their beneficial effects while moderating selected adverse effects.

Homocysteinemia is caused by deficient recycling of homocysteine back to methionine or deficient transulfuration to cysteine. Vitamins B6, B9, and B12 are involved in recycling homocysteine back to methionine, thus, a relative or absolute deficiency of any of them raises homocysteine levels. Conversely, mild to moderate homocysteinemia may be treated using any of these vitamins. Vitamin B6 also facilitates transulfuration of homocysteine to cysteine, so unlike vitamin B9 and vitamin B12 it lowers homocysteine levels through recycling as well as transulfuration. All three vitamins are well tolerated in mammals, and are available as combination drug products, such as Foltx®, Metanx® or CerefolinNAC® (Pamlab), FDA-approved combinations that are marketed for the treatment of homocysteinemia.

The betaine pathway and the vitamin B6/B9/B12-mediated pathway diverge: betaine itself provides large amounts of methyl groups, rather than re-appropriating methyl groups from other sources. Because betaine does not deplete endogenous methyl groups, its use is advantageous for conditions where the endogenous methyl pool is depleted, such as alcoholic liver disease and other liver diseases, and in niacin, nicotinamide or fibrate therapy.

Homocysteine recycling involves remethylation of homocysteine, converting it back to its precursor methionine. In most tissues, this transaction is mediated by vitamins B6, B9, and B12. First, serine hydroxy-methyltransferase (SHMT) facilitates the transfer of a methyl group from serine to tetrahydrofolate (vitamin B9), using vitamin B6 as a co-factor, yielding 5,10-CH₂-tetrahydrofolate. The latter is converted to 5-CH₃-tetrahydrofolate by methylene tetrahydrofolate reductase (MTHFR), the form of vitamin B9 that acts as a methyl donor. Next, the methylated B9 transfers the methyl group to homocysteine, facilitated by methionine synthase with vitamin B12 as a co-factor. Importantly, taking exogenous vitamin B9 does not necessarily introduce new methyl groups to the system, so ultimately homocysteine recycling depends on a repleted endogenous methyl pool. In the liver and kidneys a parallel system mediates homocysteine recycling. Betaine (also known as trimethylglycine) transfers a methyl group to homocysteine facilitated by betaine homocysteine methyltransferase (BHMT), resulting in its conversion back to methionine. Clinically, since most treatment of homocysteinemia empirically starts with vitamin therapy, betaine is also added empirically when vitamin therapy is insufficient.

The invention further includes compounds comprising niacin, a fibrate, any combination thereof, or any prodrug or analogue thereof, that is covalently bound to a methylation-enhancing or methyl-donating molecule or an analogue thereof, such as a vitamin or supplement. In one embodiment, the niacin, fibrate, any combination thereof, or analogue thereof, is covalently bound through a linker to the methylation enhancing molecule or analogue thereof. In another embodiment, the niacin, fibrate, combination thereof, or analogue thereof, is covalently bound directly to the methylation-enhancing molecule or analogue thereof. In one aspect, the compounds of the invention have a slower metabolism than niacin, and thus produce reduced flushing in the mammal. In another aspect, the methylation-enhancing component of the compounds of the invention mitigates the adverse side effect of increased homocysteine levels observed upon niacin or fibrate administering. In yet another aspect, the compounds of the invention alter lipid profiles, including raising HDL or apolipoprotein A levels in a mammal, without causing insulin resistance, reducing insulin sensitivity or promoting hepatotoxicity. Non-limiting examples of methylation-enhancing molecules contemplated within the invention include betaine (trimethylglycine), vitamin B6, vitamin B9 (folate), vitamin B12, serine and numerous compounds listed above that decrease methylation demand by providing end-products of methylation reactions (e.g., creatine).

### Methionine Metabolism

Methionine is available from diet or proteolysis, and is the precursor to S-adenosyl methionine (SAM), which acts as a methyl donor in reactions in several tissues. In donating its methyl group, SAM becomes S-adenosyl homocysteine (SAH, not shown), which is converted to homocysteine. There are two pathways to remove excessive homocysteine levels. During severe elevations of homocysteine, it may be irreversibly transulfurated into cysteine, and then eliminated in the urine. The transulfuration reaction depends on vitamin B6, and occurs in the liver, kidney, small bowel, and pancreas. More typically, homocysteine is regulated by recycling it back to methionine, through acceptance of a methyl group from another source. Most tissues achieve this by re-appropriating a methyl group from serine in a process that depends on vitamin B6. The methyl group is transferred to vitamin B9 (folate), and then to homocysteine in a process that depends on vitamin B12. The liver and the kidneys are equipped with a separate system in which the choline byproduct betaine (trimethylglycine) donates a methyl group to homocysteine, yielding methionine. Since betaine occurs naturally in common foods, a key difference in this pathway is that no endogenous methyl groups are required by this pathway. This could prove advantageous in the setting of methyl group depletion.

### Niacin

Niacin undergoes extensive first-pass metabolism in the liver and the bulk of niacin metabolism yields homocysteine. However, niacin and its metabolites are not confined to the liver, and much of radiolabeled niacin concentrates in the liver, adipose, and skin. In fact, almost every tissue in the body is primed to take up niacin (the precursor to NAD), and has the capacity to metabolize excess niacin/NAD, by a process that necessarily involves the production of homocysteine. Adipose tissue has been implicated as an important, if not the major, source of plasma homocysteine resulting from niacin metabolism, with high concentrations of nicotinamide N-methyltransferase (NNMT), the enzyme that facilitates methyl transfer from SAM to nicotinamide at the N-position, yielding 1-methylnicotinamide. Thus, adipose tissue may be a source of niacin-induced homocysteine.

Niacin itself does not consistently alter inflammatory markers during chronic therapy. In one aspect, chronic anti-inflammatory effects of niacin are counteracted by inflammatory effects of niacin-induced homocysteinemia. Reversing homocysteinemia may result in a net anti-inflammatory effect in the group that receives the homocysteine-lowering supplements. Inflammatory signal may be increased by an acute dose of immediate-release niacin before and after chronic therapy. In one aspect, the pro-inflammatory effect of niacin is attenuated or abolished by homocysteine-lowering vitamins. In another aspect, niacin-induced homocysteinemia provokes the inflammatory cascade following acute exposure to immediate-release niacin, possibly through homocysteine's ability to stimulate NFkB. In yet another aspect, homocysteine-lowering supplements inhibit homocysteine formation and in turn inhibit the inflammatory response to acute niacin administration.

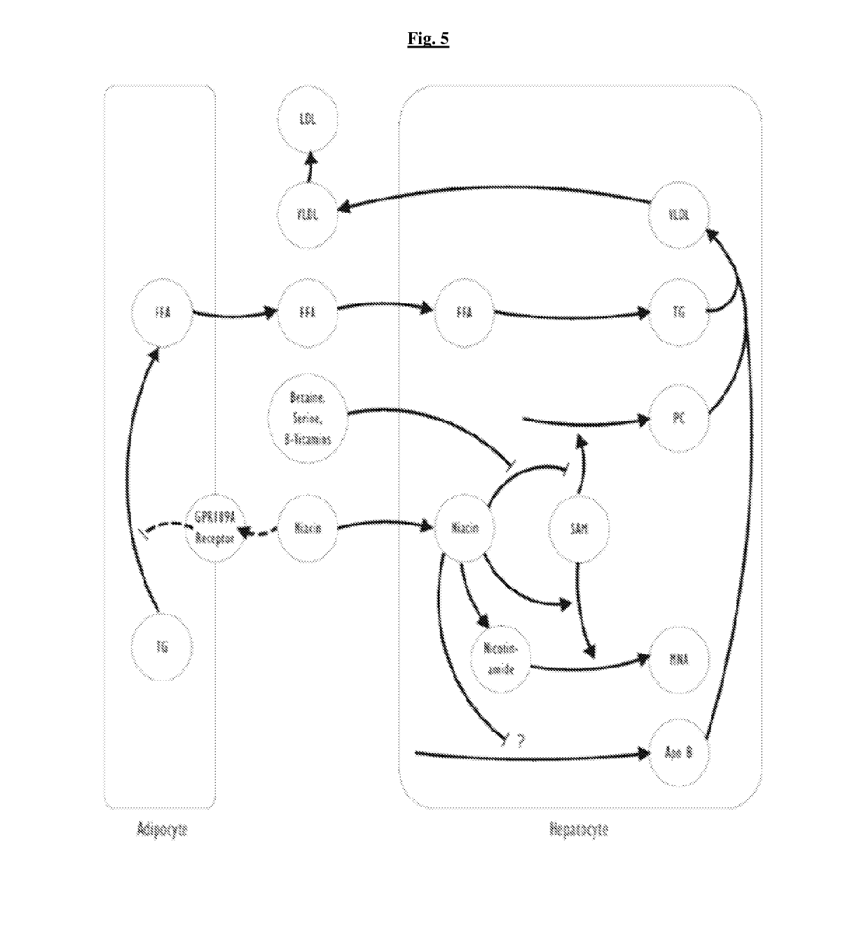

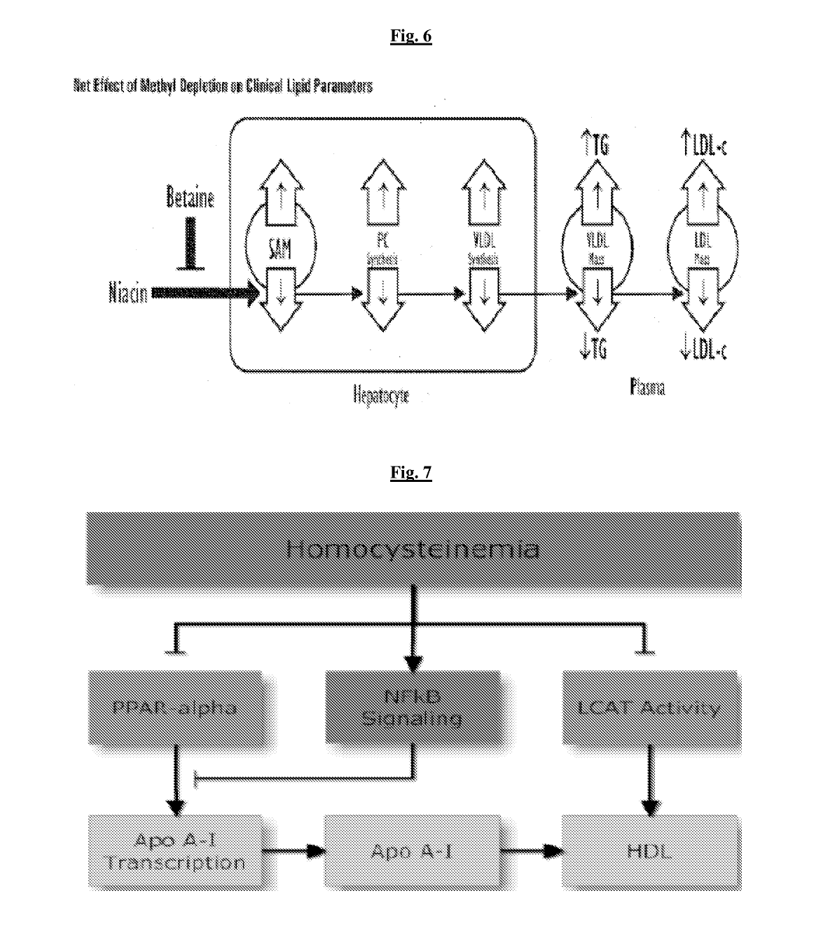

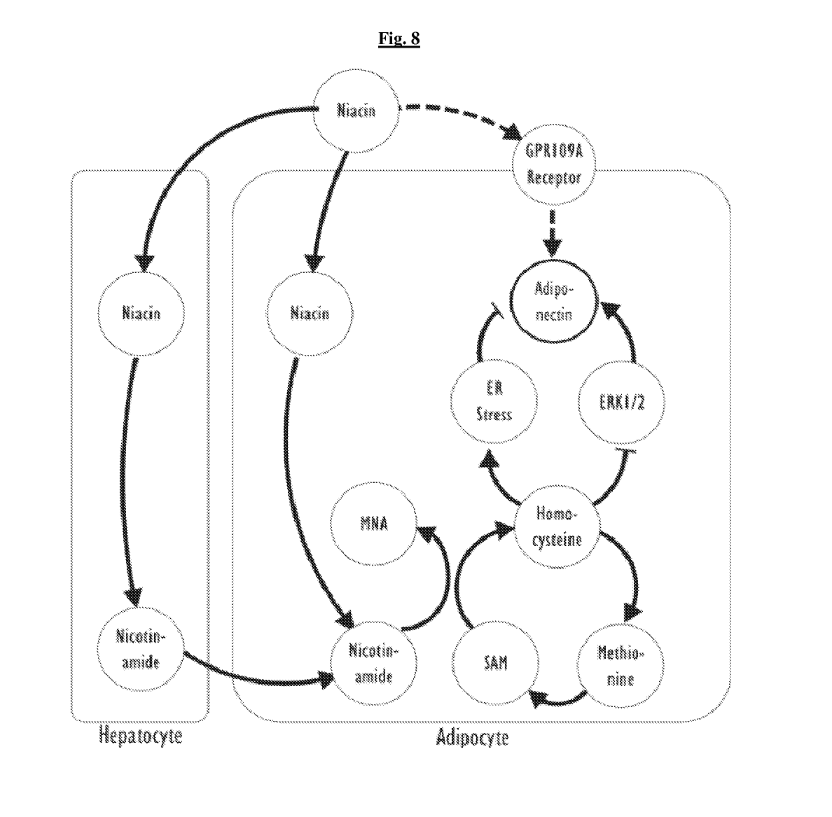

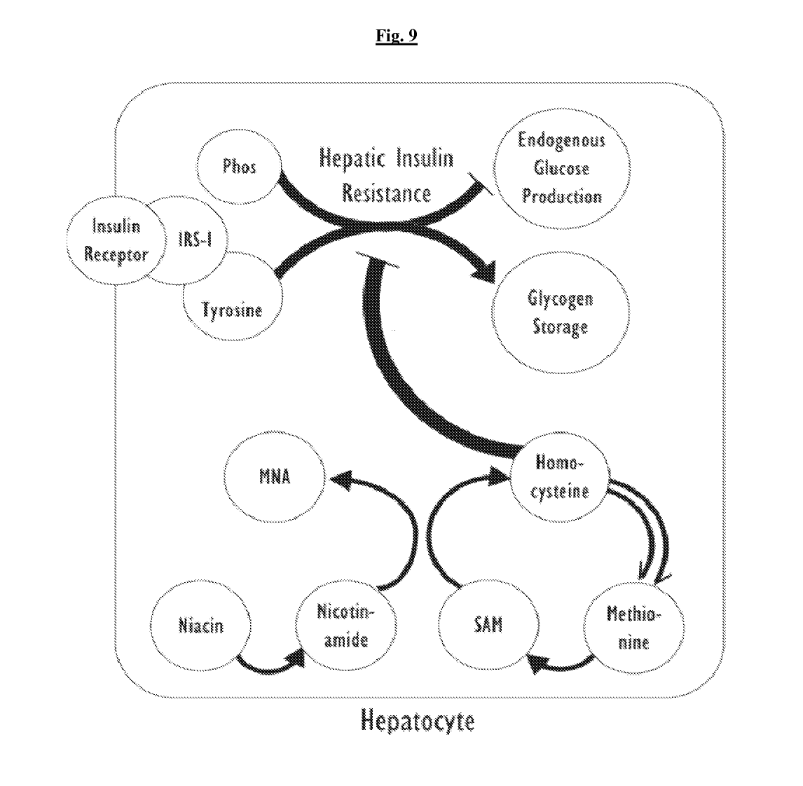

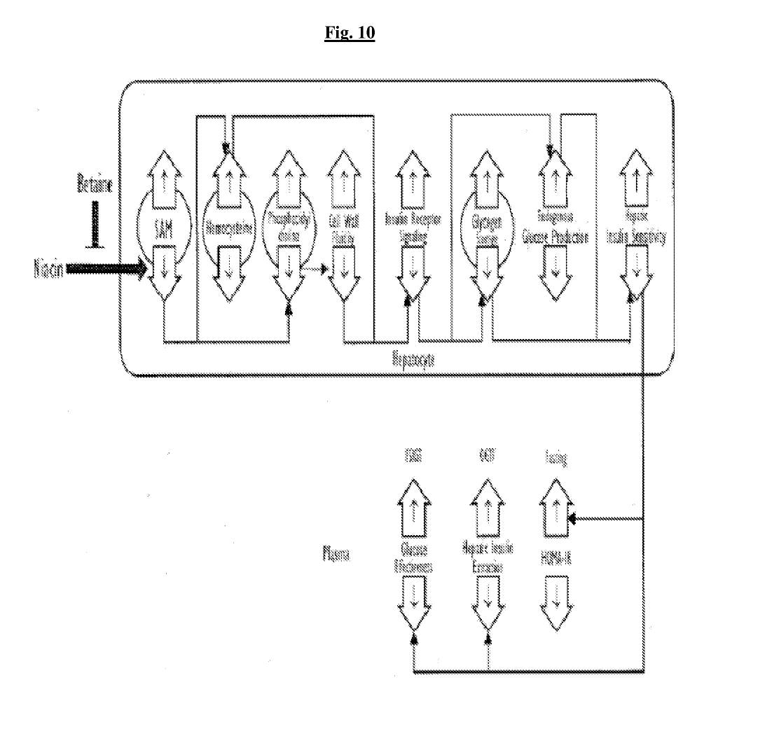

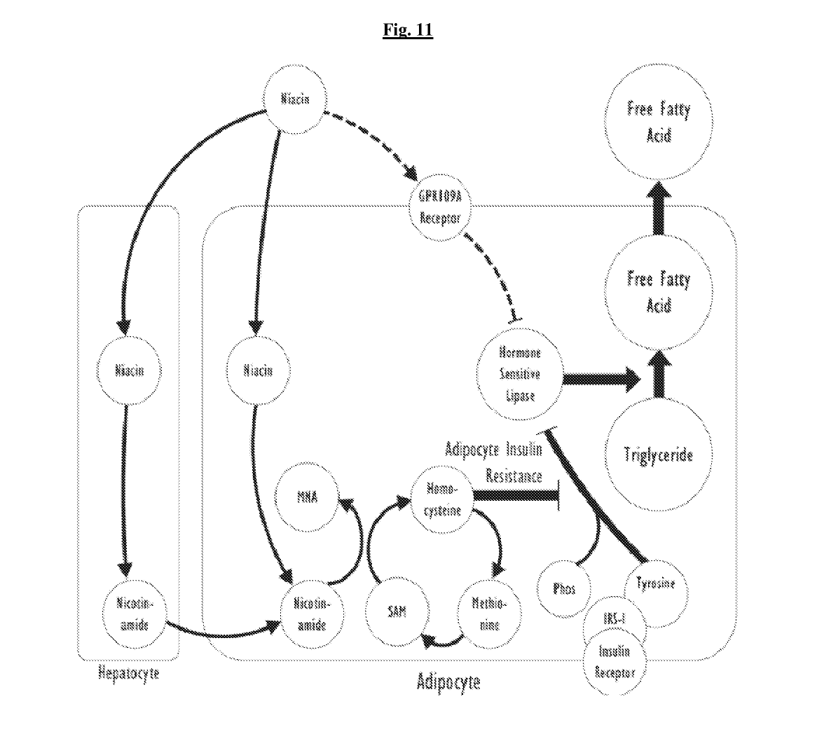

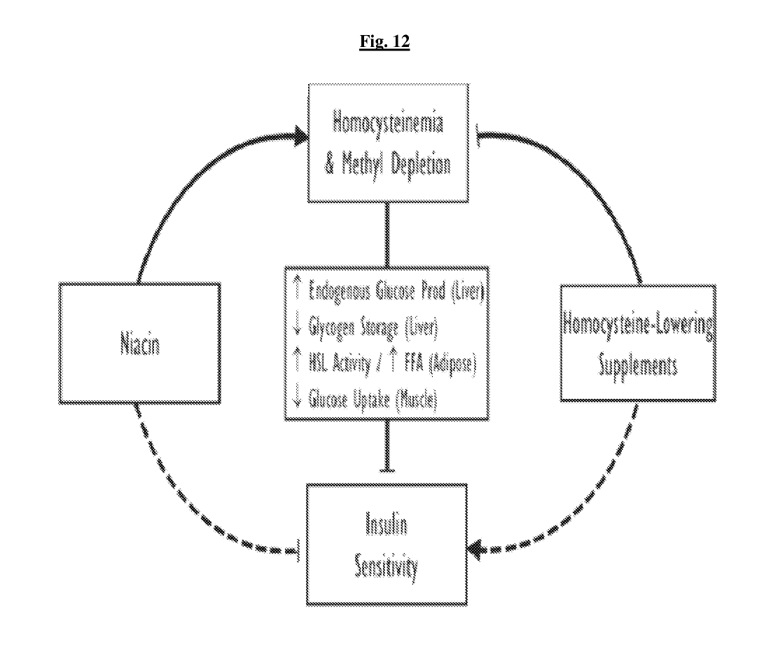

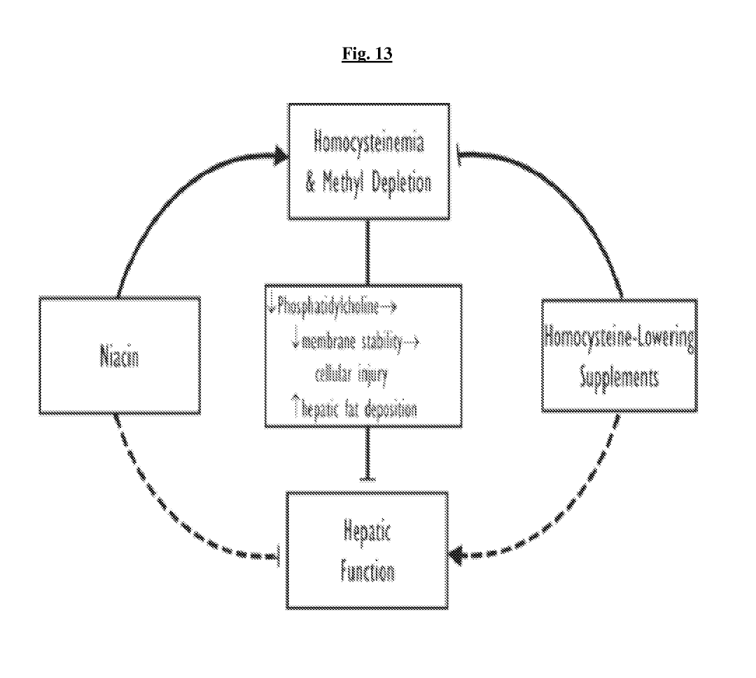

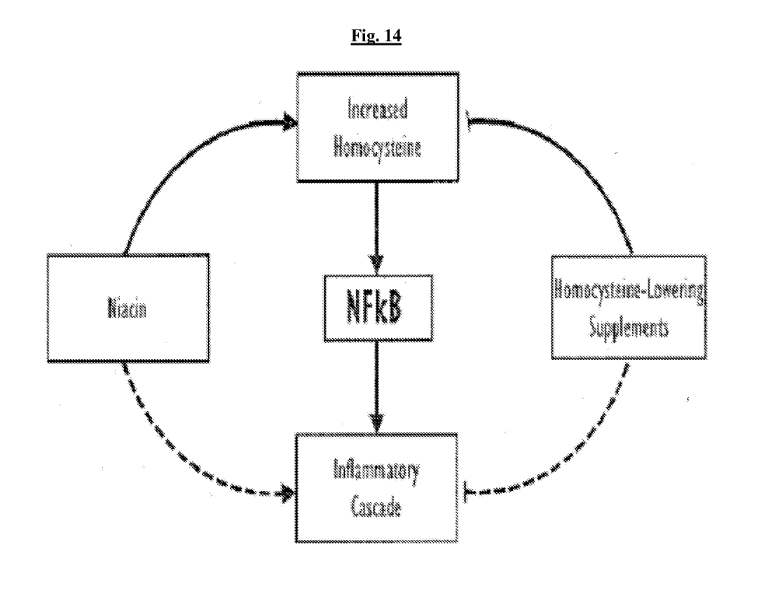

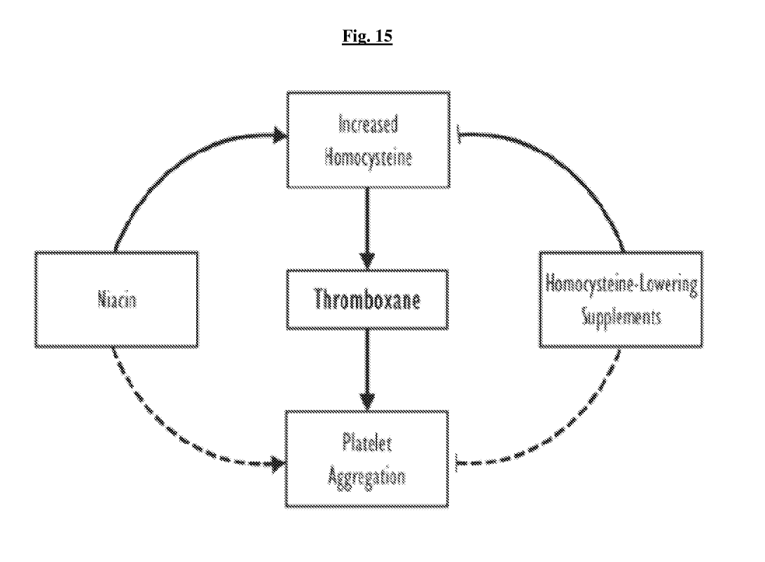

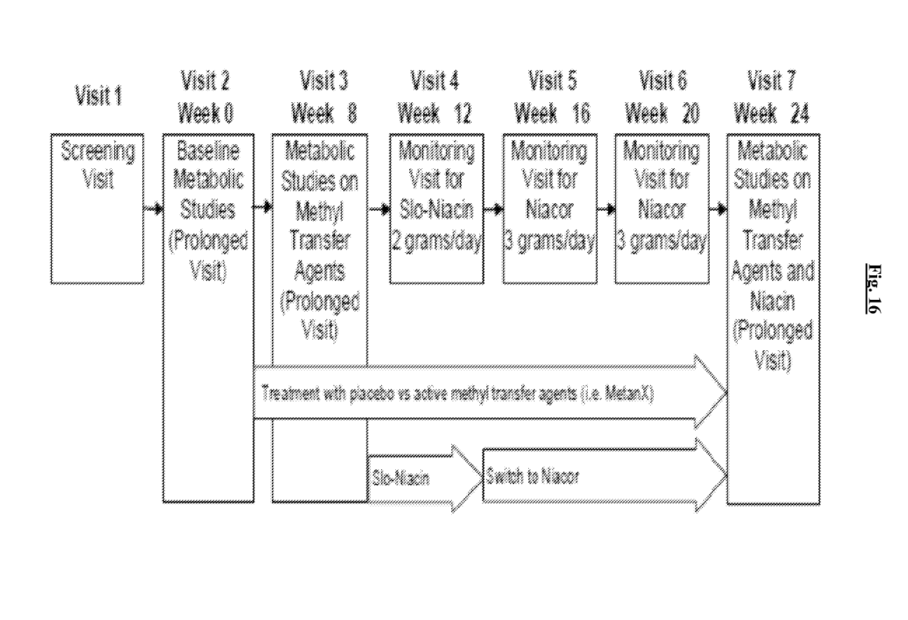

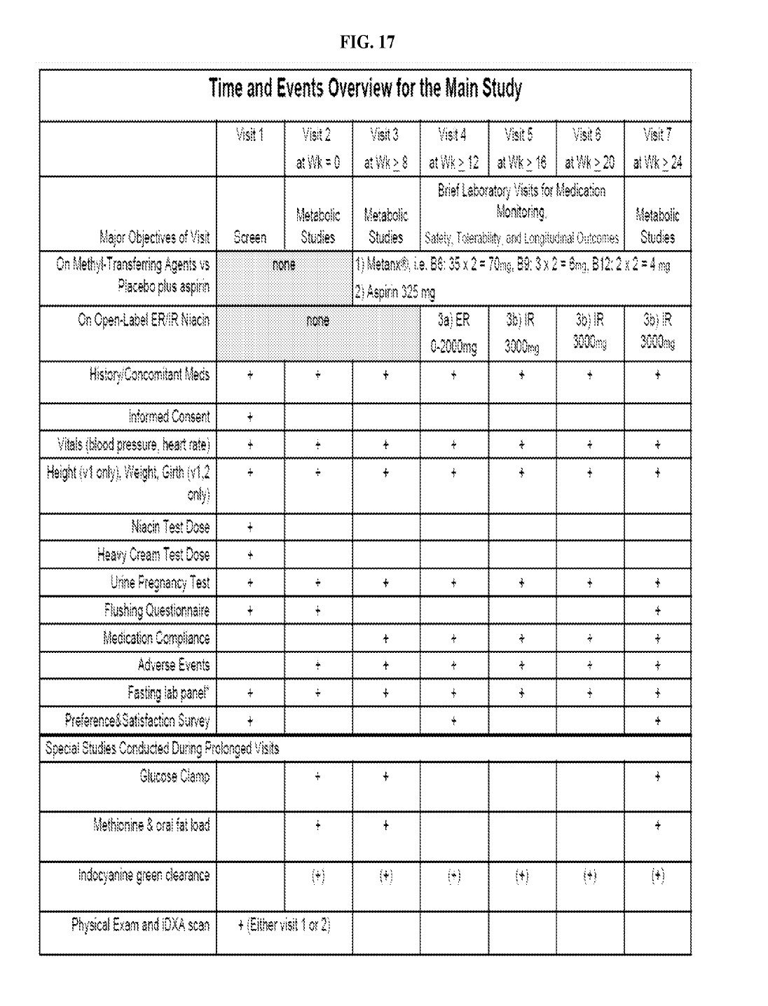

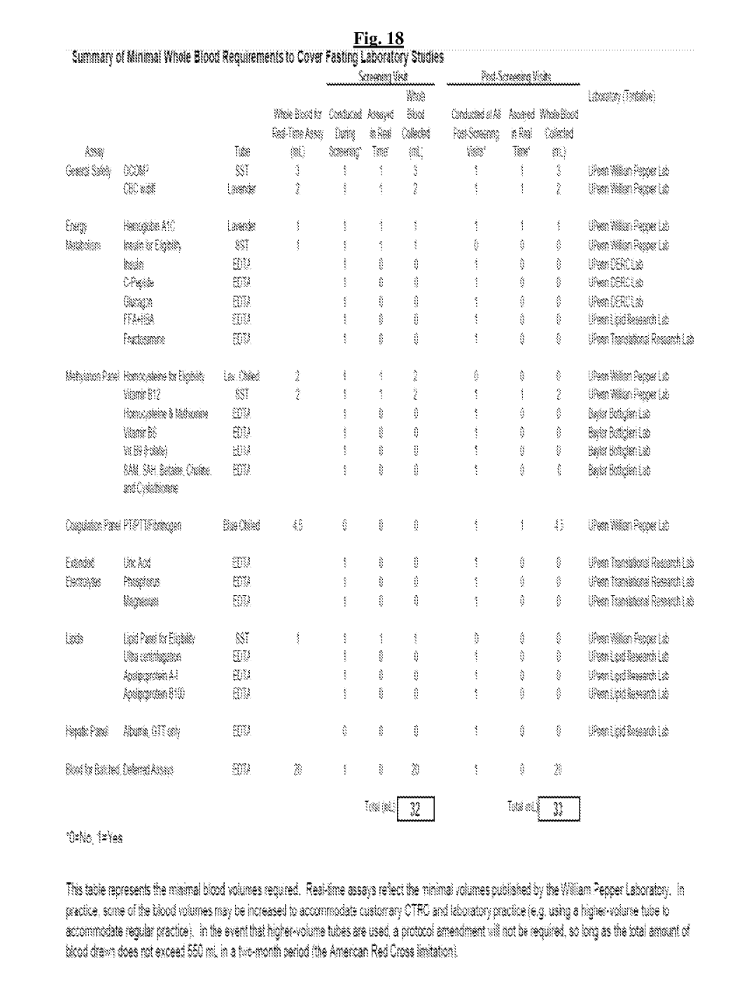

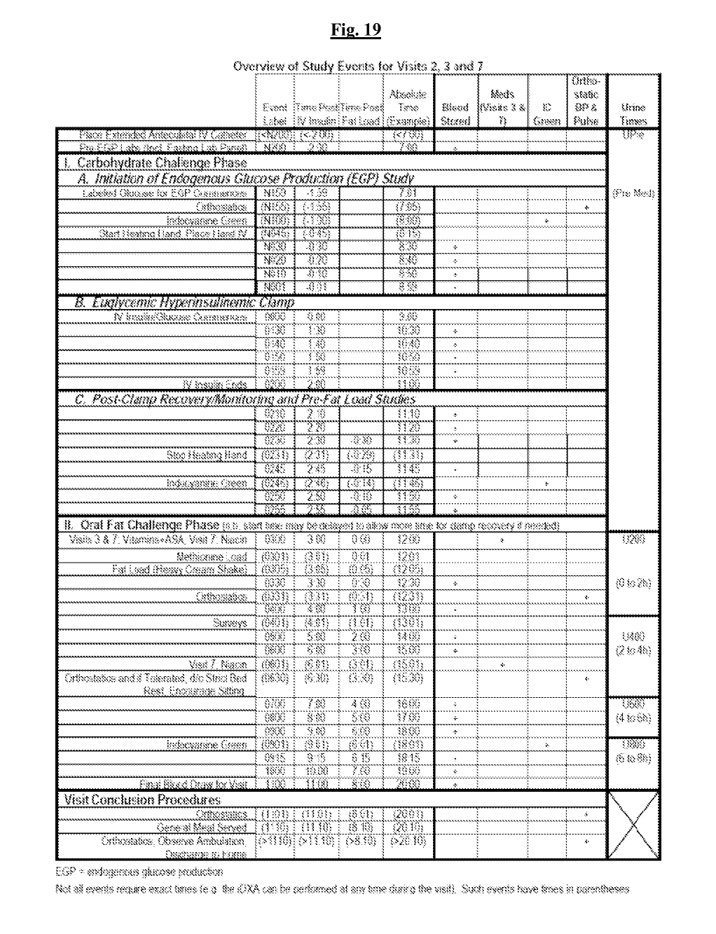

Niacin has two effects on VLDL (FIG. 5): a transient and indirect effect of FFA limitation (adipocyte panel on the left) and a durable and more direct effect of SAM and phosphatidyl choline (PC) limitation (hepatic panel on the right).

FFA Limitation:

Plasma niacin doses are elevated for several hours after a dose. During this time, niacin stimulates the GPR109A receptor on adipocytes, which inhibits lipolysis of stored adipose triglyceride (TG) from the lipid droplet, thus limiting production of FFA. Since VLDL synthesis is dependent on FFA, in the early hours after a niacin dose, the drug may limit VLDL synthesis by substrate limitation. During the FFA rebound phase, FFA is available to the liver in excess. If FFA limitation alone inhibited VLDL synthesis, VLDL synthesis should be disinhibited during FFA rebound. Instead, VLDL remains suppressed by niacin several hours after the anti-lipolytic phase has passed, and despite the several fold increase in FFA exposure. The cause of this durable suppression of VLDL is not well understood.

SAM Limitation:

The durable effect of niacin on hepatic VLDL suppression is based on SAM limitation. Niacin utilizes very large amounts of SAM for its detoxification during first and subsequent passes through the liver. Specifically, SAM is diverted to methylation of nicotinamide, yielding methyl nicotinamide (MNA). Similarly, SAM is diverted to methylation of nicotinate, yielding methyl nicotinate (trigonelline). Diversion of SAM limits synthesis of phosphatidyl choline. As the latter is a required component of VLDL, in one embodiment, SAM limitation propagates VLDL suppression initiated by FFA limitation. This would be manifest by lower plasma apolipoprotein B, triglycerides, VLDL-c, and LDL-c. Niacin inhibits PC synthesis and thereby inhibits VLDL, whereas betaine disinhibits PC synthesis and thereby disinhibits VLDL. If SAM limitation is significant, betaine restores SAM availability, and niacin loses a portion of its ability to lower VLDL. On the other hand, in the absence of niacin therapy, in one embodiment SAM is replete and PC synthesis is normal. Accordingly, betaine is not expected to affect VLDL in the absence of niacin, so betaine is not expected to exert a significant effect on triglycerides or LDL-c during betaine monotherapy. Niacin does not lower triglycerides of LDL-c to the same extent when given with betaine, suggesting this pathway plays a role in niacin's mechanism of action.

The net effect of niacin on the hepatocyte is to deplete the SAM pool. In turn, this decreases the rates of phosphatidyl choline (PC) and VLDL synthesis by the liver. This results in decreased prevalence of VLDL and its by-product LDL in plasma. Clinically, this may be detected by decreased triglyceride (TG) and LDL-c. Betaine dosed in a quantity equimolar to niacin prevents depletion of the SAM pool, and thereby attenuates these effects. The degree to which betaine disrupts triglyceride and LDL-lowering serves as a rough guide to the importance of this pathway (if any) to niacin's mechanism of action. In other words, if betaine has no effect, this pathway is not involved in niacin's benefits on atherogenic dyslipidemia. Partial dependence on SAM deficiency suggests a multifactorial mechanism of action.

Niacin-induced homocysteinemia promotes niacin-induced insulin resistance by disrupting insulin signaling. Excess hepatic homocysteine stimulates hepatic endogenous glucose production and inhibits glycogen storage, both of which manifest as a decrease in hepatic insulin sensitivity. Excess adipose homocysteine inhibits insulin inhibition of hormone-sensitive lipase (HSL). In turn, disinhibited HSL results in increased nocturnal or niacin-stimulated free fatty acids (FFA), which prolongs disrupted insulin signaling in numerous tissues. Since niacin concentrates in the liver and adipose, intracellular homocysteine levels are highest in those tissues, disrupting the insulin signaling of those tissues more than peripheral tissues. Insulin signaling is disrupted in muscle, either directly by homocysteinemia or indirectly by way of FFA elevation. Methyl depletion promotes insulin resistance by limiting production of phosphatidylcholine. The latter is a key component of organelle and cell wall membranes, and deficiency of phosphatidylcholine may disrupt signaling by cell-surface receptors such as the insulin receptor by altered membrane fluidity.

Niacin-induced homocysteinemia provokes niacin-induced hepatotoxicity by disrupting hepatic phosphatidylcholine synthesis due to mass diversion of methyl groups toward niacin metabolism Impaired phosphatidylcholine destabilizes hepatic membranes (e.g., endoplasmic reticulum or the cell wall), which itself could precipitate cellular injury. Since phosphatidyl-choline is a required element of VLDL, limited supply reduces VLDL secretion, trapping free fatty acids and triglycerides in the liver. This helps explains fatty liver seen from niacin or nicotinamide in pre-clinical models. The net effect is that niacin impairs hepatic function by way of excess hepatic homocysteine. Homocysteine-lowering supplements inhibit homocysteine and in turn disinhibit niacin's deleterious effect on hepatic function, thus improving hepatic function.

### Fibrates

Fibrates, also known as fibric acid derivatives, activate the peroxisome proliferators-activated receptor (PPAR)-alpha, altering the expression of genes involved in lipid metabolism and leading to an increase in plasma HDL levels and reduction in TG levels. Fibrates also reduce LDL levels and the proportion of atherogenenic small, dense LDL particles to a lower extent (de Graaf et al., 1993, Arter. & Thromb. 13:712-9). Preferred fibrates are those agents that have been marketed, most preferred are fenofibrate, bezafibrate, and gemfibrozil, or a pharmaceutically acceptable salt thereof.

### Hepatic Insulin Resistance

Disrupted methylation metabolism may cause hepatic insulin resistance, which may be determined by at least the following techniques: the homeostatic model assessment, version 2 (HOMA2, Wallace et al., 2004, Diabetes Care 27(6):1487-95) or revised quantitative insulin sensitivity check index (rQUICKI), as a fasting measure of insulin resistance and reflecting hepatic insulin sensitivity; determination of hepatic glucose production by radiolabeled or stable isotope-labeled glucose infusion; and measurement of insulin and c-peptide to estimate fractional hepatic insulin extraction (FHIE), a test of insulin sensitivity specific to the liver (Faber et al., 1981, J. Clin. Endocrinol. Metab. 53(3):618-21). Taking these measurements in the context of acute and acute-on-chronic niacin-dosing allows for the assessment of correlation between hepatic insulin sensitivity and the free fatty acid rebound.

A more sensitive method for measuring hepatic function on- and off-drug therapy comprises the use of indocyanine green. This compound is exclusively cleared by the liver, and provides an integrated marker of hepatic perfusion, parenchymal function, and biliary excretion. This compound has been used extensively to study the pharmacodynamics effects of medications (Abraldes et al., 2009, Gastroent. 136(5):1651-8; Edwards et al., 1987, Eur. J. Clin. Pharmacol. 32(5):481-4; Feely & Wood, 1983, Br. J. Clin. Pharmacol. 5(1):109-11; Feely & Wood, 1983, Clin. Pharmacol. Ther. 33(1):91-4) and to study the influence of different physiologic states (Faybik & Hetz, 2006, Transplant Proc. 38(3):80 1-2) on hepatic function, especially hepatic blood flow. Indocyanine green clearance has been used to infer hepatic function. Previously, the dye levels had to be measured in plasma, necessitating frequent blood draws and laboratory studies to derive a clearance curve. Recently, a non-invasive measurement technique has been developed, so that drug levels can be determined without the need for sampling of plasma (Alander et al., 2012, Int'l J. Biomed. Imag. Vol. 2012, Article ID 940585). Indocyanine green absorbs at 805 nm Because there are no other endogenous pigmented compounds that strongly absorb that wavelength, the concentration of indocyanine green may be determined by optical densitometry. Determination of the green signal of indocyanine green involves placing a non-invasive optical probe on the pinna of the ear or the finger, and is not uncomfortable.

The densitometry technique allows real-time data capture of a continuous curve of the decay characteristics. This allows a robust determination of hepatic function and due to its extremely brief half-life and excellent safety profile the test can be safely conducted up to 20 times a day in the same mammal. The test has a wide applicable age range, from newborn infants to the very elderly. Indocyanine green analysis provides a more sensitive test of hepatic function than the currently available clinical laboratory tests.

### Lipid Profile

One way to analyze the dyslipidemia (or hyperlipidemia) profile of a patient is to classify it according to the Fredrickson classification. This widely accepted classification is based on the pattern of lipoproteins on electrophoresis or ultracentrifugation (Frederickson & Lee, 1965, Circulation 31: 321-7), being adopted by the World Health Organization (WHO). This classification does not directly account for HDL. The general classes of the Fredrickson classification are shown in Table 2.

TABLE 2
Fredrickson classification of hyperlipoproteinemia

Lipid Elevation

| Type | Lipoprotein elevated | Major | Minor |
|---|---|---|---|
| I (rare) | chylomicrons | TG | ↑ [symbol] C |
| IIa | LDL | C | — |
| IIb | LDL, VLDL | C | TG |
| III (rare) | IDL | C, TG | — |
| IV | VLDL | TG | ↑ [symbol] C |
| V (rare) | chylomicrons, | TG | ↑ [symbol] C |
| | VLDL | | |

C = cholesterol;
TG = triglycerides;
LDL = low density lipoprotein;
VLDL = very low density lipoprotein;
IDL = intermediate density lipoprotein

Type I hyperlipoproteinemia is a form of hyperlipoproteinemia associated with deficiencies of lipoprotein lipase.

Type II hyperlipoproteinemia, by far the most common form, is further classified into Type IIa and Type IIv, depending mainly on whether there is elevation in the TG levels in addition to LDL levels. Type IIa (also known as familial hypercholesterolemia) may be sporadic (due to dietary factors), polygenic, or truly familial as a result of a mutation either in the LDL receptor gene on chromosome 19 (0.2% of the population) or the ApoB gene (0.2%). The familial form is characterized by tendon xanthoma, xanthelasma and premature cardiovascular disease. The incidence of this disease is about 1 in 500 for heterozygotes, and 1 in 1,000,000 for homozygotes. Type IIb has high VLDL levels, due to overproduction of substrates, including triglycerides, acetyl CoA, and an increase in B-100 synthesis, and may also be caused by the decreased clearance of LDL. The prevalence in the population is 10%.

Type III hyperlipoproteinemia is characterized by high chylomicrons levels and IDL (intermediate density lipoprotein) levels. Also known as broad beta disease or dysbetalipoproteinemia, the most common cause for this form is the presence of ApoE E2/E2 genotype. It is characterized by cholesterol-rich VLDL (β-VLDL), and prevalence is 0.02% of the population.

Type IV hyperlipoproteinemia (familial) is associated with high TG levels. It is also known as hypertriglyceridemia (or pure hypertriglyceridemia). According to the NCEP-ATP III definition of high triglyceride levels (>200 mg/dl), prevalence is about 16% of adult population.

Type V hyperlipoproteinemia (endogenous) is very similar to type I, but with high VLDL levels in addition to chylomicrons, and also associated with glucose intolerance and hyperuricemia.

Two unclassified forms of hyperlipoproteinemia are extremely rare: hypo-alpha lipoproteinemia and hypo-beta lipoproteinemia (prevalence 0.01-0.1%).

The levels of each individual lipid, lipoprotein or related molecule may be associated with a low, medium or high risk of cardiovascular disease. Therefore, the levels of these lipids, lipoproteins and related molecules in an individual should be analyzed individually to determine whether the individual has an overall low, medium or high risk of cardiovascular disease, and whether the individual would benefit from changes in life style and/or use of medications. The following figures are provided by the National Cholesterol Education program (NCEP) of the National Institutes of Health (NIH), and are based on the Third Report of the Expert Panel on Detection, Evaluation, and Treatment of High Blood Cholesterol in Adults (Adult Treatment Panel III, or ATP III), as shown in Table 3.

TABLE 3
Lipid profile thresholds as defined by ATP III.

| Total cholesterol | Desirable: | <200 | mg/dL |
|---|---|---|---|
| | | <5.2 | mmol/L |
| | Borderline high: | 200-239 | mg/dL |
| | | 5.2-6.2 | mmol/L |
| | High: | 240 mg/dL or higher | |
| | | 6.2 mmol/L or higher | |
| HDL cholesterol | High (desirable): | >60 | mg/dL |
| | | >1.6 | mmol/L |
| | Acceptable: | 40-60 | mg/dL |
| | | 1.0-1.6 | mmol/L |
| | Low (undesirable): | <40 | mg/dL |
| | | <1.0 | mmol/L |
| Total cholesterol-to-HDL | Desirable: | 5:1 or less | |
| ratio | | | |
| LDL cholesterol | Undesirable: | >5:1 | |
| | Optimal: | <100 | mg/dL |
| | | <2.6 | mmol/L |
| | Near optimal: | 100-129 | mg/dL |
| | | 2.6-3.3 | mmol/L |
| | Borderline high: | 130-159 | mg/dL |
| | | 3.4-4.1 | mmol/L |
| | High: | 160-189 | mg/dL |
| | | 4.1-4.9 mmol/L or higher | |
| | Very high: | 190 mg/dL or higher | |
| | | 4.9 mmol/L or higher | |
| VLDL cholesterol | Optimal: | 30 mg/dL or less | |
| | | 0.78 mmol/L or less | |
| Triglycerides | Normal: | <150 | mg/dL |
| | | <1.7 | mmol/L |
| | Borderline high: | 150-199 | mg/dL |
| | | 1.7-2.3 | mmol/L |
| | High: | 200-499 | mg/dL |
| | | 2.3-5.6 | mmol/L |

| | |
|---|---|
| Very high: | 500 mg/dL or higher |
| | 5.6 mmol/L or higher |

Furthermore, as specified by the American Heart Association, high hsCRP levels correlate with higher risks of heart attack. In fact, the risk for heart attack in people in the upper third of hsCRP levels has been determined to be twice that of those whose hsCRP levels are in the lower third. Studies have also found an association between sudden cardiac death, peripheral arterial disease and hsCRP. The current literature reports that, if the hsCRP levels are lower than 1.0 mg/l, a person has a low risk of developing cardiovascular disease. If the hsCRP levels are between 1.0 and 3.0 mg/l, a person has an average risk. If the hsCRP levels are higher than 3.0 mg/l, a person is at high risk.

### Compositions of the Invention

The invention includes a pharmaceutical composition comprising a lipid-managing medication and a methylation enhancing supplement, wherein the lipid-managing medication comprises niacin or a fibrate, or a prodrug, analogue or metabolite thereof.

In one embodiment, the methylation enhancing supplement comprises betaine, serine, methionine, s-adenosyl methionine, choline, phosphatidylcholine, creatine, cysteamine, cysteine, N-acetylcysteine, silymarin, alpha lipoic acid, melatonin, vitamin B5 (pantothenic acid), pantethine, silymarin, whey protein, vitamin B6, vitamin B9, vitamin B12, a salt thereof, or any combinations thereof. In another embodiment, the supplement is not silymarin if the lipid-managing medication comprises niacin or a prodrug, analogue or metabolite thereof. In yet another embodiment, the lipid-managing medication and the methylation enhancing supplement are co-formulated for administration to the mammal. In yet another embodiment, the lipid-managing medication is in a controlled release formulation. In yet another embodiment, the lipid-managing medication is in an intermediate-release or extended-release formulation.

In one embodiment, the composition comprises about 2 grams to about 6 grams of the lipid-managing medication. In another embodiment, the composition comprises at least one amount selected from the group consisting of about 50 mg of vitamin B6, about 5 mg of vitamin B9, about 4 μg of vitamin B12, and about 2 grams to about 6 grams of betaine.

### Methods of the Invention

The invention includes a method of reducing or preventing the increase of triglyceride levels in a mammal in need thereof. The method comprises administering to the mammal a therapeutically effective amount of a pharmaceutically acceptable composition comprising a lipid-managing medication, wherein the mammal is further administered a methylation enhancing supplement, thereby reducing or preventing the increase of triglyceride levels in the mammal.

The invention also includes a method of increasing or preventing the decrease of HDL levels in a mammal in need thereof. The method comprising administering to the mammal a therapeutically effective amount of a pharmaceutically acceptable composition comprising a lipid-managing medication, wherein the mammal is further administered a methylation enhancing supplement, thereby increasing or preventing the decrease of HDL levels in the mammal.

In one embodiment, the lipid-managing medication comprises niacin, a fibrate, or a prodrug, analogue or metabolite thereof.

In one embodiment, the methylation enhancing supplement comprises betaine, serine, methionine, s-adenosyl methionine, choline, phosphatidylcholine, creatine, cysteamine, cysteine, N-acetylcysteine, alpha lipoic acid, melatonin, silymarin, vitamin B5, pantethine, whey protein, vitamin B6, vitamin B9, vitamin B12, any salts thereof, or any combinations thereof. In another embodiment, the methylation enhancing supplement is not silymarin if the lipid-managing medication comprises niacin or a prodrug, analogue or metabolite thereof.

In one embodiment, the lipid-managing medication and the methylation enhancing supplement are separately administered to the mammal. In another embodiment, the lipid-managing medication and the methylation enhancing supplement are concomitantly administered to the mammal. In yet another embodiment, the lipid-managing medication and the methylation enhancing supplement are co-formulated for administration to the mammal. In yet another embodiment, the lipid-managing medication is in a controlled release formulation. In yet another embodiment, the lipid-managing medication is in an intermediate-release or extended-release formulation.

In one embodiment, the mammal develops less insulin resistance than when the mammal is administered a similar dose of lipid-managing medication and is not administered the methylation enhancing supplement. In another embodiment, the mammal develops less tachyphylaxis than when the mammal is administered a similar dose of lipid-managing medication and is not administered the methylation enhancing supplement. In yet another embodiment, the mammal develops less hepatotoxicity than when the mammal is administered a similar dose of lipid-managing medication and is not administered the methylation enhancing supplement.

In one embodiment, the mammal is administered from about 2 grams to about 6 grams of the lipid-managing medication daily. In another embodiment, the mammal is administered at least one daily dose selected from the group consisting of about 50 mg of vitamin B6, about 5 mg of vitamin B9, about 4 μg of vitamin B12, and about 2 grams to about 6 grams of betaine. In yet another embodiment, the mammal is a human.

The invention also includes a method of reducing or preventing the increase of triglyceride levels in a mammal in need thereof. The method comprises administering to the mammal a therapeutically effective amount of a pharmaceutically acceptable composition comprising a methylation enhancing supplement, thereby reducing or preventing the increase of triglyceride levels in the mammal.

The invention also includes a method of increasing or preventing the decrease of HDL levels in a mammal in need thereof. The method comprises administering to the mammal a therapeutically effective amount of a pharmaceutically acceptable composition comprising a methylation enhancing supplement, thereby increasing or preventing the decrease of HDL levels in the mammal.

The invention also includes a method of treating or preventing insulin resistance in a mammal in need thereof. The method comprises administering to the mammal a therapeutically effective amount of a pharmaceutically acceptable composition comprising a methylation enhancing supplement, thereby treating or preventing insulin resistance in the mammal.

In one embodiment, the methylation enhancing supplement comprises betaine, serine, methionine, s-adenosyl methionine, choline, phosphatidylcholine, creatine, cysteamine, cysteine, N-acetylcysteine, alpha lipoic acid, melatonin, vitamin B5, pantethine, silymarin, whey protein, vitamin B6, vitamin B9, vitamin B12, a salt thereof, or any combinations thereof. In another embodiment, the methylation enhancing supplement is in a controlled release formulation. In yet another embodiment, the methylation enhancing supplement is in an intermediate-release or extended-release formulation. In yet another embodiment, the mammal is a human.

### Administration/Dosage

Administration of the compositions of the present invention to a patient to be treated may be carried out using known procedures, at dosages and for periods of time effective to inhibit a lipoprotein abnormality in the patient. An effective amount of the therapeutic compound necessary to achieve a therapeutic effect may vary according to factors such as the state of the disease or disorder in the patient, the age, sex, and weight of the patient, and the ability of the therapeutic compound to control or revert the lipoprotein abnormality or insulin resistance in the patient. Dosage regimens may be adjusted to provide the optimum therapeutic response. For example, several divided doses may be administered daily or the dose may be proportionally reduced as indicated by the exigencies of the therapeutic situation. A non-limiting example of an effective dose range for a therapeutic compound useful within the methods of the invention (e.g., niacin or a fibrate) is between about 2 and about 250 mg/kg of body weight/per day. One of ordinary skill in the art would be able to study the relevant factors and make the determination regarding the effective amount of the therapeutic compound without undue experimentation.

Actual dosage levels of the active ingredients in the pharmaceutical compositions useful within the methods of the invention may be varied so as to obtain an amount of the active ingredient that is effective to achieve the desired therapeutic response for a particular patient, composition, and mode of administration, without being toxic to the patient.

In particular, the selected dosage level depends upon a variety of factors including the activity of the particular compound employed, the time of administration, the rate of excretion of the particular compound being employed, the duration of the treatment, other drugs, compounds or materials used in combination with the particular compound employed, the age, sex, weight, condition, general health and prior medical history of the patient being treated, and like factors well, known in the medical arts.

A medical doctor, e.g., physician or veterinarian, having ordinary skill in the art may readily determine and prescribe the effective amount of the pharmaceutical composition required. For example, the physician or veterinarian could start doses of the compounds useful within the methods of the invention employed in the pharmaceutical composition at levels lower than that required in order to achieve the desired therapeutic effect and gradually increase the dosage until the desired effect is achieved.

The regimen of administration may affect what constitutes an effective amount. The therapeutic formulations may be administered to the patient either prior to or after the onset of a lipoprotein abnormality. Further, several divided dosages, as well as staggered dosages, may be administered daily or sequentially, or the dose may be continuously infused, or may be a bolus injection. Further, the dosages of the therapeutic formulations may be proportionally increased or decreased as indicated by the exigencies of the therapeutic or prophylactic situation.

In particular embodiments, it is especially advantageous to formulate compositions in dosage unit form for ease of administration and uniformity of dosage. Dosage unit form as used herein refers to physically discrete units suited as unitary dosages for the patients to be treated; each unit containing a predetermined quantity of therapeutic compound calculated to produce the desired therapeutic effect in association with the required pharmaceutical vehicle. The specification for the dosage unit forms of the invention are dictated by and directly dependent on (a) the unique characteristics of the therapeutic compound and the particular therapeutic effect to be achieved, and (b) the limitations inherent in the art of compounding/formulating such a therapeutic compound for the treatment of a lipoprotein abnormality in patients.

In another embodiment, the compositions useful within the methods of the invention are formulated using one or more pharmaceutically acceptable excipients. The excipients are selected from any one or more of starch, sugar, cellulose, diluent, granulating agent, lubricant, binder, disintegrating agent, wetting agent, emulsifier, coloring agent, release agent, coating agent, sweetening agent, flavoring agent, perfuming agent, preservative, antioxidant, plasticizer, gelling agent, thickener, hardener, setting agent, suspending agent, surfactant, humectant, carrier, and stabilizer, or any combination thereof.

In other embodiments, the compositions of the invention are administered to the patient in dosages that range from one to five times per day or more. In yet other embodiments, the compositions of the invention are administered to the patient in range of dosages that include once every day, every two, days, every three days to once a week, once every two weeks, etc. It will be readily apparent to one skilled in the art that the frequency of administration of the various combination compositions varies from individual to individual depending on many factors including, but not limited to, age, disease or disorder to be treated, gender, overall health, and other factors. Thus, the invention should not be construed to be limited to any particular dosage regime and the precise dosage and composition to be administered to any patient will be determined by the attending physical taking all other factors about the patient into account.

In one embodiment, the dose of niacin or any prodrug, analogue or metabolite thereof is the molar equivalent to a nicotinic acid amount ranging between about 250 mg and about 6,000 mg, and any and all whole or partial increments there between. In another embodiment, niacin is administered in forms that enhance cellular uptake, for example, by binding a niacin molecule or a niacin prodrug, analogue or metabolite to a fat-soluble compound or any other compound designed to enhance uptake. In yet another embodiment, niacin or any niacin prodrug, analogue or metabolite is administered in a formulation using other techniques to enhance uptake. In such embodiments, the therapeutic dose of niacin or its prodrug, analogue or metabolite may be considerably lower than the aforementioned molar equivalent doses (i.e., lower than a molar equivalent to a nicotinic acid mass ranging from 250 mg to 6,000 mg), and such therapeutic doses are included within the present invention. The invention also includes any other novel niacin-related medications, including any approved and/or marketed dose range for niacin or one of its prodrugs, analogues or metabolites.

In one non-limiting embodiment, the doses of fibrates contemplated within the invention are as follows. The dose of gemfibrozil or any prodrug, analogue or metabolite thereof is the molar equivalent to a daily amount of gemfibrozil ranging between 600 and 1,200 mg, and any and all whole or partial increments there between. The dose of fenofibrate or any prodrug, analogue or metabolite thereof is the molar equivalent to a daily amount of fenofibrate ranging between 30 and 200 mg, and any and all whole or partial increments there between. The dose of fenofibric acid (e.g., Trilipix) or any prodrug, analogue or metabolite thereof is the molar equivalent to a daily amount of fenofibric acid ranging between 45 and 135 mg, and any and all whole or partial increments there between. The dose of clofibrate or any prodrug, analogue or metabolite thereof is the molar equivalent to a daily amount of clofibrate ranging between 500 and 2,000 mg, and any and all whole or partial increments there between. The dose of bezafibrate or any prodrug, analogue or metabolite thereof is the molar equivalent to a daily amount of bezafibrate ranging between 200 and 400 mg, and any and all whole or partial increments there between. The dose of ciprofibrate or any prodrug, analogue or metabolite thereof is the molar equivalent to a daily amount of ciprofibrate ranging between 25 and 100 mg, and any and all whole or partial increments there between. The dose of etofibrate or any prodrug, analogue or metabolite thereof is the molar equivalent to a daily amount of etofibrate ranging between 100 and 500 mg, and any and all whole or partial increments there between. The invention also contemplates any approved and/or marketed dose range for a fibrate, or any prodrug, analogue or metabolite thereof.

In one non-limiting embodiment, the dose of betaine (trimethylglycine) or any prodrug, analogue or metabolite thereof is the molar equivalent to a daily amount of betaine ranging from about 200 mg to about 6,000 mg, and any and all whole or partial increments there between.

In one non-limiting embodiment, the dose of serine or any prodrug, analogue or metabolite thereof is the molar equivalent to a daily amount of serine ranging from about 150 mg to about 6,000 mg, and any and all whole or partial increments there between.

In one non-limiting embodiment, the dose of methionine or any prodrug, analogue or metabolite thereof is the molar equivalent to a daily amount of methionine ranging from about 250 mg to about 8,000 mg, and any and all whole or partial increments there between.

In one non-limiting embodiment, the dose of s-adenosyl methionine or any prodrug, analogue or metabolite thereof is the molar equivalent to a daily amount of s-adenosyl methionine ranging from about 750 mg to about 20,000 mg, and any and all whole or partial increments there between.

In one non-limiting embodiment, the dose of choline or any prodrug, analogue or metabolite thereof is the molar equivalent to a daily amount of choline ranging from about 150 mg to about 6,000 mg, and any and all whole or partial increments there between.

In one non-limiting embodiment, the dose of phosphatidylcholine or any prodrug, analogue or metabolite thereof is the molar equivalent to a daily amount of phosphatidylcholine ranging from about 1,000 mg to about 20,000 mg, and any and all whole or partial increments there between.

In one non-limiting embodiment, the dose of nicotinic acid N-methylbetaine (trigonelline) or any prodrug, analogue or metabolite thereof is the molar equivalent to a daily amount of nicotinic acid N-methylbetaine ranging from about 250 mg to about 8,000 mg, and any and all whole or partial increments there between.

In one non-limiting embodiment, the dose of creatine or any prodrug, analogue or metabolite thereof is the molar equivalent to a daily amount of creatine ranging from about 250 mg to about 25,000 mg, and any and all whole or partial increments there between.

In one non-limiting embodiment, the dose of cysteamine or any prodrug, analogue or metabolite thereof is the molar equivalent to a daily amount of cysteamine ranging from about 50 mg to about 2,000 mg, and any and all whole or partial increments there between.

In one non-limiting embodiment, the dose of cysteine or any prodrug, analogue or metabolite thereof is the molar equivalent to a daily amount of cysteine ranging from about 250 mg to about 3,000 mg, and any and all whole or partial increments there between.

In one non-limiting embodiment, the dose of N-acetylcysteine or any prodrug, analogue or metabolite thereof is the molar equivalent to a daily amount of N-acetylcysteine ranging from about 250 mg to about 2,400 mg, and any and all whole or partial increments there between.

In one non-limiting embodiment, the dose of silymarin or any component thereof or any prodrug, analogue or metabolite thereof is the molar equivalent to a daily amount of silymarin ranging from about 50 mg to about 1,500 mg, and any and all whole or partial increments there between.

In one non-limiting embodiment, the dose of alpha lipoic acid or any prodrug, analogue or metabolite thereof is the molar equivalent to a daily amount of alpha lipoic acid ranging from about 50 mg to about 1,500 mg, and any and all whole or partial increments there between.

In one non-limiting embodiment, the dose of melatonin or any prodrug, analogue or metabolite thereof is the molar equivalent to a daily amount of melatonin ranging from about 0 mg to about 30 mg, and any and all whole or partial increments there between.

In one non-limiting embodiment, the dose of vitamin B5 (pantothenic acid) or any prodrug, analogue or metabolite thereof is the molar equivalent to a daily amount of vitamin B5 ranging from about 10 mg to about 1,200 mg, and any and all whole or partial increments there between.

In one non-limiting embodiment, the dose of pantethine or any prodrug, analogue or metabolite thereof is the molar equivalent to a daily amount of pantethine ranging from about 200 mg to about 1,500 mg, and any and all whole or partial increments there between.

In one non-limiting embodiment, the dose of whey protein or any prodrug, analogue or metabolite thereof is the molar equivalent to a daily amount of whey protein ranging from about 250 mg to about 30,000 mg, and any and all whole or partial increments there between.

In one non-limiting embodiment, the dose of vitamin B6 or any prodrug, analogue or metabolite thereof is the molar equivalent to a daily amount of vitamin B6 ranging from about 35 mg to about 100 mg, and any and all whole or partial increments there between.

In one non-limiting embodiment, the dose of vitamin B9 or any prodrug, analogue or metabolite thereof is the molar equivalent to a daily amount of vitamin B9 ranging from about 3 mg to about 10 mg, and any and all whole or partial increments there between.

In one embodiment, the dose of vitamin B12 or any prodrug, analogue or metabolite thereof is the molar equivalent to a daily amount of vitamin B12 ranging from about 2 mg to about 10 mg, and any and all whole or partial increments there between.

In one non-limiting embodiment, the compositions of the present invention may be useful in combination with one or more additional compounds useful for treating a lipoprotein abnormality. These additional compounds may comprise compounds of the present invention or compounds, e.g., commercially available compounds, known to treat, prevent, or reduce the symptoms of a lipoprotein abnormality.

### Formulations for Administration

In another embodiment, the present invention is directed to a packaged pharmaceutical composition comprising a container holding a therapeutically effective amount of a composition of the invention, alone and in combination with a second pharmaceutical agent; and instructions for using the composition of the invention to treat, prevent, or reduce one or more symptoms of a lipoprotein abnormality in a patient.

Granulating techniques are well known in the pharmaceutical art for modifying starting powders or other particulate materials of an active ingredient. The powders are typically mixed with a binder material into larger permanent free-flowing agglomerates or granules referred to as a “granulation.” For example, solvent-using “wet” granulation processes are generally characterized in that the powders are combined with a binder material and moistened with water or an organic solvent under conditions resulting in the formation of a wet granulated mass from which the solvent must then be evaporated.

Melt granulation generally consists in the use of materials that are solid or semi-solid at room temperature (i.e., having a relatively low softening or melting point range) to promote granulation of powdered or other materials, essentially in the absence of added water or other liquid solvents. The low melting solids, when heated to a temperature in the melting point range, liquefy to act as a binder or granulating medium. The liquefied solid spreads itself over the surface of powdered materials with which it is contacted, and on cooling, forms a solid granulated mass in which the initial materials are bound together. The resulting melt granulation may then be provided to a tablet press or be encapsulated for preparing the oral dosage form. Melt granulation improves the dissolution rate and bioavailability of an active (i.e., drug) by forming a solid dispersion or solid solution.

U.S. Pat. No. 5,169,645 discloses directly compressible wax-containing granules having improved flow properties. The granules are obtained when waxes are admixed in the melt with certain flow improving additives, followed by cooling and granulation of the admixture. In certain embodiments, only the wax itself melts in the melt combination of the wax(es) and additives(s), and in other cases both the wax(es) and the additives(s) will melt.

In a further embodiment, the present invention relates to a method of manufacturing a multi-layer tablet comprising a layer providing for the delayed release of the compositions of the invention, and a further layer providing for the immediate release of an anti-ulcer agent such as a proton pump inhibitor, an H2-receptor antagonist, and/or sucralfate. Using a wax/pH-sensitive polymer mix, a gastric insoluble composition may be obtained in which the active ingredient is entrapped, ensuring its delayed release.

Formulations may be employed in admixtures with conventional excipients, i.e., pharmaceutically acceptable organic or inorganic carrier substances suitable for oral, parenteral, nasal, intravenous, subcutaneous, enteral, or any other suitable mode of administration, known to the art. Suitable pharmaceutically acceptable carriers include but are not limited to water, salt solutions, alcohols, gum arabic, vegetable oils, benzyl alcohols, polyethylene glycols, gelate, carbohydrates such as lactose, amylose or starch, magnesium stearate talc, silicic acid, viscous paraffin, perfume oil, fatty acid monoglycerides and diglycerides, pentaerythritol fatty acid esters, hydroxymethylcellulose, polyvinylpyrrolidone, etc. The pharmaceutical preparations may be sterilized and if desired mixed with auxiliary agents, e.g., lubricants, preservatives, stabilizers, wetting agents, emulsifiers, salts for influencing osmotic pressure buffers, coloring, flavoring and/or aromatic substances and the like. They may also be combined where desired with other active agents, e.g., other analgesic agents. For oral application, particularly suitable are tablets, dragees, liquids, drops, suppositories, or capsules, caplets and gel caps. The compositions intended for oral use may be prepared according to any method known in the art and such compositions may contain one or more agents selected from the group consisting of inert, non-toxic pharmaceutically excipients which are suitable for the manufacture of tablets. Such excipients include, for example an inert diluent such as lactose; granulating and disintegrating agents such as cornstarch; binding agents such as starch; and lubricating agents such as magnesium stearate. The tablets may be uncoated or they may be coated by known techniques for elegance or to delay the release of the active ingredients. Formulations for oral use may also be presented as hard gelatin capsules wherein the active ingredient is mixed with an inert diluent.

The term “container” includes any receptacle for holding the pharmaceutical composition. For example, in one embodiment, the container is the packaging that contains the pharmaceutical composition. In other embodiments, the container is not the packaging that contains the pharmaceutical composition, i.e., the container is a receptacle, such as a box or vial that contains the packaged pharmaceutical composition or unpackaged pharmaceutical composition and the instructions for use of the pharmaceutical composition. Moreover, packaging techniques are well known in the art. It should be understood that the instructions for use of the pharmaceutical composition may be contained on the packaging containing the pharmaceutical composition, and as such the instructions form an increased functional relationship to the packaged product. However, it should be understood that the instructions may contain information pertaining to the compound's ability to perform its intended function, e.g., treating, preventing, or reducing one or more gastrointestinal disorder in a patient.

Another embodiment of the invention is a pharmaceutical composition comprising a therapeutically effective amount of a composition of the invention and a pharmaceutically acceptable carrier.

The pharmaceutically acceptable carrier may be a solvent or dispersion medium containing, for example, water, ethanol, polyol (for example, glycerol, propylene glycol, and liquid polyethylene glycol, and the like), suitable mixtures thereof, and vegetable oils. The proper fluidity may be maintained, for example, by the use of a coating such as lecithin, by the maintenance of the required particle size in the case of dispersion and by the use of surfactants. Prevention of the action of microorganisms may be achieved by various antibacterial and antifungal agents, for example, parabens, chlorobutanol, phenol, ascorbic acid, thimerosal, and the like. In many cases, it will be preferable to include isotonic agents, for example, sugars, sodium chloride, or polyalcohols such as mannitol and sorbitol, in the composition. Prolonged absorption of the injectable compositions may be brought about by including in the composition an agent which delays absorption, for example, aluminum monostearate or gelatin. In one embodiment, the pharmaceutically acceptable carrier is not DMSO alone.

The compounds for use in the invention may be formulated for administration by any suitable route, such as for oral or parenteral, for example, transdermal, transmucosal (e.g., sublingual, lingual, (trans)buccal, (trans)urethral, vaginal (e.g., trans- and perivaginally), (intra)nasal and (trans)rectal), intravesical, intrapulmonary, intraduodenal, intragastrical, intrathecal, subcutaneous, intramuscular, intradermal, intra-arterial, intravenous, intrabronchial, inhalation, and topical administration, as long as adequate systemic exposure is achieved.

Suitable compositions and dosage forms include, for example, tablets, capsules, caplets, pills, gel caps, troches, dispersions, suspensions, solutions, syrups, granules, beads, transdermal patches, gels, powders, pellets, magmas, lozenges, creams, pastes, plasters, lotions, discs, suppositories, liquid sprays for nasal or oral administration, dry powder or aerosolized formulations for inhalation, compositions and formulations for intravesical administration and the like. It should be understood that the formulations and compositions that would be useful in the present invention are not limited to the particular formulations and compositions that are described herein.

### Oral Administration

For oral administration, the compounds may be in the form of tablets or capsules prepared by conventional means with pharmaceutically acceptable excipients such as binding agents (e.g., polyvinylpyrrolidone, hydroxypropylcellulose or hydroxypropylmethylcellulose); fillers (e.g., cornstarch, lactose, microcrystalline cellulose or calcium phosphate); lubricants (e.g., magnesium stearate, talc, or silica); disintegrates (e.g., sodium starch glycollate); or wetting agents (e.g., sodium lauryl sulphate). If desired, the tablets may be coated using suitable methods and coating materials such as OPADRY® film coating systems available from Colorcon, West Point, Pa. (e.g., OPADRY® OY Type, OYC Type, Organic Enteric OY-P Type, Aqueous Enteric OY-A Type, OY-PM Type and OPADRY® White, 32K18400). Liquid preparation for oral administration may be in the form of solutions, syrups or suspensions. The liquid preparations may be prepared by conventional means with pharmaceutically acceptable additives such as suspending agents (e.g., sorbitol syrup, methyl cellulose or hydrogenated edible fats); emulsifying agent (e.g., lecithin or acacia); non-aqueous vehicles (e.g., almond oil, oily esters or ethyl alcohol); and preservatives (e.g., methyl or propyl p-hydroxy benzoates or sorbic acid).

### Parenteral Administration

For parenteral administration, the compounds may be formulated for injection or infusion, for example, intravenous, intramuscular or subcutaneous injection or infusion, or for administration in a bolus dose and/or continuous infusion. Suspensions, solutions or emulsions in an oily or aqueous vehicle, optionally containing other formulatory agents such as suspending, stabilizing and/or dispersing agents may be used.

### Controlled Release Formulations and Drug Delivery Systems

In certain embodiments, the formulations of the present invention may be, but are not limited to, short-term, rapid-offset, as well as controlled, for example, sustained release, delayed release and pulsatile release formulations.

The term sustained release is used in its conventional sense to refer to a drug formulation that provides for gradual release of a drug over an extended period of time, and that may, although not necessarily, result in substantially constant blood levels of a drug over an extended time period. The period of time may be as long as a month or more and should be a release which is longer that the same amount of agent administered in bolus form.

For sustained release, the compounds may be formulated with a suitable polymer or hydrophobic material which provides sustained release properties to the compounds. As such, the compounds for use the method of the invention may be administered in the form of microparticles for example, by injection or in the form of wafers or discs by implantation.

In a preferred embodiment of the invention, the compositions of the invention are administered to a patient, alone or in combination with another pharmaceutical agent, using a sustained release formulation.

The term delayed release is used herein in its conventional sense to refer to a drug formulation that provides for an initial release of the drug after some delay following drug administration and that mat, although not necessarily, includes a delay of from about 10 minutes up to about 12 hours.

The term pulsatile release is used herein in its conventional sense to refer to a drug formulation that provides release of the drug in such a way as to produce pulsed plasma profiles of the drug after drug administration.

The term immediate release is used in its conventional sense to refer to a drug formulation that provides for release of the drug immediately after drug administration.

As used herein, short-term refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes and any or all whole or partial increments there between after drug administration after drug administration.

As used herein, rapid-offset refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes, and any and all whole or partial increments there between after drug administration.

Those skilled in the art recognizes, or is able to ascertain using no more than routine experimentation, numerous equivalents to the specific procedures, embodiments, claims, and examples described herein. Such equivalents were considered to be within the scope of this invention and covered by the claims appended hereto. For example, it should be understood, that modifications in reaction conditions, including reaction times, reaction size/volume, and experimental reagents, such as solvents, catalysts, pressures, atmospheric conditions, e.g., nitrogen atmosphere, and reducing/oxidizing agents, etc., with art-recognized alternatives and using no more than routine experimentation, are within the scope of the present application.

It is to be understood that wherever values and ranges are provided herein, e.g., in ages of patient populations, dosages, and blood levels, all values and ranges encompassed by these values and ranges, are meant to be encompassed within the scope of the present invention. Moreover, all values that fall within these ranges, as well as the upper or lower limits of a range of values, are also contemplated by the present application.

The following examples further illustrate aspects of the present invention. However, they are in no way a limitation of the teachings or disclosure of the present invention as set forth herein.

### EXAMPLES

The invention is now described with reference to the following Examples. These Examples are provided for the purpose of illustration only, and the invention is not limited to these Examples, but rather encompasses all variations that are evident as a result of the teachings provided herein.

### Materials:

Unless otherwise noted, all starting materials and resins were obtained from commercial suppliers and used without purification.

### Example 1

### Efficacy of Methylation Enhancing Supplements Alone

Therapy is initiated with the methylation enhancing supplements two months prior to niacin initiation, to assess the primary effect of the supplements on study outcomes without superimposition of niacin therapy. In one embodiment, this provides a separate assessment of the primary effects of the supplements on idiopathic insulin resistance versus niacin-induced insulin resistance. In another embodiment, this provides a separate assessment of the primary effects of the supplements on dyslipidemia versus niacin-treated dyslipidemia. In another embodiment, two months' worth of methylation enhancing supplements is sufficient to ensure that the treated group starts niacin with a full complement of methyl transferring vitamins. In another embodiment, providing the methyl-transferring vitamins a head start shortens the time for a differential to develop between the groups when niacin is initiated.

### Example 2

### Clinical Study Comprising Niacin Therapy

The study is a randomized, double-blinded, placebo-controlled, parallel arm, clinical pilot experiment to test the concept that niacin-induced homocysteinemia contributes to adverse effects of niacin. The study compares supplements that replete the methyl pool and reduce homocysteine to placebo (i.e., methylation enhancing supplements) added to niacin therapy. Subjects are screened to determine eligibility.

Summary:

This study assesses how methylation enhancing supplements improve hepatic insulin sensitivity compared to placebo during chronic niacin therapy. Hepatic insulin sensitivity is determined primarily by endogenous glucose production, and secondarily by fasting Fractional Hepatic Insulin Extraction (FHIE) and homeostasis model assessment of insulin resistance (HOMA-IR).

Insulin sensitivity of other tissues, namely, peripheral insulin sensitivity, is assessed by glucose infusion rate on euglycemic hyperinsulinemic clamp, and adipose insulin sensitivity is assessed by suppression of free fatty acids during the clamp.

This study further assesses how methylation enhancing supplements augment the rise in plasma HDL cholesterol compared to placebo during niacin therapy. This is achieved using fasting samples following a 12 hour overnight fast, and secondarily by incremental area under the curve following an oral fat load. As corroboratory assays, apolipoprotein A-I, the major protein of HDL and related biomarkers known to mediate changes in HDL cholesterol and apolipoprotein A-I (e.g., adiponectin, inflammatory markers) are also assessed.

This study further assesses how methylation enhancing supplements disrupt the drop in plasma triglycerides compared to placebo during niacin therapy. This is achieved using fasting samples following a 12 hour overnight fast, and secondarily by incremental area under the curve of the triglycerides following an oral fat load. As corroboratory assays, VLDL cholesterol, apolipoprotein B-100, and LDL cholesterol are also assessed.

Efficacy of Homocysteine Suppression:

This study assesses whether methylation enhancing supplements prevent niacin-induced homocysteinemia compared to placebo during chronic niacin therapy. This is determined primarily by post-methionine-load homocysteine and secondarily by fasting homocysteine.

Lipoprotein:

This study assesses whether methylation enhancing supplements attenuate the drop in plasma triglycerides, VLDL cholesterol, apolipoprotein B-100, and LDL cholesterol compared to placebo during niacin therapy. This is determined primarily by fasting samples following a 12-hour overnight fast and secondarily by incremental area under the curve of the lipids following an oral fat load.

This study further assesses whether methylation enhancing supplements augment the rise in plasma apolipoprotein A-I and HDL cholesterol compared to placebo during niacin therapy (i.e., supplements prevent tachyphylaxis). This is determined primarily by fasting samples following a 12 hour overnight fast.

This study further assesses whether methylation enhancing supplements increase adiponectin compared to placebo during chronic niacin therapy.

Insulin Resistance:

This study assesses whether supplements that augment the hepatic methyl pool and reduce homocysteine improve hepatic, adipose, and peripheral insulin sensitivity compared to placebo independent from and during chronic niacin therapy.

In one embodiment, insulin resistance is measured based on endogenous glucose production monitored using an isotopically labeled glucose (such as, but not limited to, deuterated or tritiated glucose) technique. The endogenous glucose production is measured thrice, prior to all therapies, after vitamins or placebo alone, and at the end of the study on niacin plus vitamins or placebo. As corroboratory markers, fasting Factional Hepatic Insulin Extraction (FHIE), and fasting glucose, insulin, c-peptide, and FFA are assayed on each subject's batched samples to derive fasting indices (e.g., HOMA2-IR, HOMA2-S, and rQUICKI). Because these are fasting tests, they are assessed at the brief monitoring visits in addition to the three prolonged study visits, to better assess the time course of changes. Because multiple tissues are implicated in niacin-induced insulin resistance, peripheral insulin resistance is also evaluated with the euglycemic hyperinsulinemic clamp, and adipose sensitivity is evaluated by assaying fatty acids during the clamp.

In another embodiment, the study assesses whether supplements that augment the hepatic methyl pool and reduce homocysteine suppress the free fatty acid rebound in proportion to measures of niacin-induced insulin resistance during chronic niacin therapy. Fatty acid rebound is determined by fasting plasma free fatty acids following a 12 hour overnight fast, but more importantly, after a provocative acute challenge with 1 gram immediate-release niacin. In one embodiment, the test of free fatty acid rebound is immediately followed by an oral glucose load to assess whether supplementation increases fractional hepatic insulin extraction (FHIE) by the molar ratio of c-peptide to insulin areas under the curve, and does so in proportion to improved FFA metabolism.

High-Density Lipoprotein:

The lipid-related objective of the study is assessed by fasting plasma HDL-c at all post-screen visits. This is assayed using the ultracentrifugation technique, the reference method for HDL-c. Plasma is stored until a given subject completes participation, and then the lipids for that subject are assayed together to minimize laboratory variability. The ultracentrifugation panel also provides triglycerides, VLDL-c, and LDL-c. In addition, apolipoprotein A-I and apolipoprotein B are measured by spectrophotometry on an automated chemistry analyzer using Roche reagents. The primary lipid results are initially analyzed, and the latter are assayed as a corroboratory measure if appropriate. In addition to fasting visits, postprandial HDL levels are evaluated at the three prolonged visits. Apolipoproteins may also be assayed as corroboratory markers.

Non-High-Density Lipoprotein:

The study further focuses on non-high-density lipoproteins, primarily fasting triglycerides and secondarily VLDL-c, apolipoprotein B-100, and LDL-c. These are measured using the ultracentrifugation technique and spectrophotometry by the same techniques described elsewhere herein. Postprandial triglycerides are measured at the three prolonged visits as with HDL.

Hepatic Function:

This study further assesses whether supplements that augment the hepatic methyl pool and reduce homocysteine improve clinical tests of hepatic function compared to placebo during niacin therapy. This is primarily by transaminases (ALT, AST, ALP, and gamma GTP) and secondarily by albumin, bilirubin, and tests of coagulation.

This study further assesses whether supplements that augment the hepatic methyl pool and reduce homocysteine improve hepatic clearance of indocyanine green as a high-sensitivity pharmacodynamic indicator of hepatic parenchymal function and clearance through the biliary system. This is determined primarily by plasma disappearance rate (PDR) and secondarily by the retention rate extrapolated to 15 minutes (R15).

Inflammation:

This study further assesses whether supplements that augment the hepatic methyl pool and reduce homocysteine suppresses the inflammatory cascade arising from acute exposure to niacin. This is determined primarily by acute changes in TNF-alpha and secondarily by changes in IL-6, absolute neutrophil count, and hsCRP following a challenge with immediate-release niacin.

Thrombosis:

This study further assesses whether supplements that augment the hepatic methyl pool and reduce homocysteine mitigate indicators of platelet activation. This is determined primarily by acute changes in urinary thromboxane metabolite levels and secondarily by changes in platelet aggregation.

Molecular Effects at the Tissue Level:

This study further assesses whether supplements that augment the hepatic methyl pool and reduce homocysteine reduce adipose homocysteine mass and mRNA expression of genes encoding proteins related to phenotypic outcomes, such as NNMT, adiponectin, and IL-6.

Time-Dependent Effects of Niacin:

This study further assesses whether acute flushing response to a provocative dose of niacin diminishes with chronic therapy. This is determined by comparing indices of flushing in response to niacin over time. The physical stigmata of flushing is determined objectively by investigator-determined measurements, primarily, the Flux Index on laser Doppler flowmetry, and secondarily, change in redness on colorimetry and change in malar thermal circulation index on thermometry. Flushing perception is determined subjectively, by participant-rated symptom scoring. In one embodiment, physical stigmata and perception of flushing significantly decreases during chronic niacin therapy.

This study further assesses whether chronic therapy with niacin moderates the transient increase in plasma bilirubin associated with acute niacin exposure.

This study further assesses whether chronic therapy with niacin lowers plasma phosphorous, describing the time course of the onset and durability of this finding.

This study further assesses whether chronic therapy with niacin lowers platelet count, describing the time course of the onset and durability of this finding.

Long-term durability of the above outcomes is studied by encouraging subjects to participate in an optional extension study. After the final visit of the main study, the dose of niacin is maintained at 3 grams daily, but now all participants receive methylation-enhancing supplements. It is expected that those previously randomized to placebo supplements will achieve parity with those previously randomized to active supplements, and that the latter will not experience any diminution of the expected benefits of the active supplements. Given the complexity of the main study, the dose-ranging extension is optional so that the extension would not become a deterrent to recruitment.

The study interventions may be summarized as:
(a) Chronic daily therapy: Randomized, double-blinded therapy with B vitamins for at least 24 weeks;
   For the first 8 weeks,
      1) Metanx® or matching placebo (one tablet twice daily, with each tablet providing vitamin B6: 35 mg, vitamin B9: 3 mg, and vitamin B12: 2 mg)
      2) open-label aspirin 325 mg once daily (in preparation for niacin therapy)
   For the next 16 weeks at minimum (up to 4 additional weeks if needed for niacin accommodation),
      1) Metanx®
      2) aspirin
      3) Background open-label niacin (Vitamin B3) therapy:
         a) Dose Titration Phase (4 wks): Slo-Niacin® 500 to 2000 mg daily;
         b) Maintenance Phase (12 wks): Niacor® 1000 mg thrice daily (i.e. 3000 mg daily).
(b) Acute Pharmacodynamic Challenges:
   Euglycemic hyperinsulinemic clamp: insulin, glucose, isotopically labeled glucose;
   Oral fat tolerance test: Heavy cream dosed by weight, with lactase enzyme;
   Post-methionine load homocysteine (amino acid challenge): L-methionine 0.1 g/kg;
   Optional hepatic parenchymal function and biliary clearance: indocyanine green 0.5 mg/kg body weight for each determination.

### General Inclusion Criteria:

Healthy subjects between the ages of 21 and 75 years of age with at least one feature of atherogenic dyslipidemia. Overweight: BMI>30 or increased abdominal girth (non-Asian men ≧40 inches, women ≧35 inches; Asian men ≧35 inches, women ≧31 inches).
If not overweight: HOMA ≧2.0
If not overweight and HOMA <2.0: HOMA ≧1.5 and (either glucose ≧100 mg/dL or HgbA1c≧5.7%).

TABLE 4

| For subjects not on statin | For subjects on stable statin** therapy |
|---|---|
| HDL < 60 mg/dL | HDL < 63 mg/dl |
| -or- TG > 150 | -or- TG > 130 |
| -or- Apo B/LDL-c ≧ 0.9 | -or- Apo B/LDL-c ≧ 0.9 |

**Statins include the following:

| | | |
|---|---|---|
| Atorvastatin (Lipitor ®) | Pitavastatin | Rosuvastatin |
| | (Livalo ®) | (Crestor ®) |
| Fluvastatin (Lescol ®) | Pravastatin | Simvastatin |
| | (Pravachol ®) | (Zocor ®) |

| | |
|---|---|
| Lovastatin (Mevacor ®) | Red Yeast Rice |

The following statin combination drugs would necessitate a washout and
switch to statin monotherapy because the non-statin component is
exclusionary, as discussed below:

| | |
|---|---|
| Lovastatin + Niacin (Advicor ®) | Simvastatin + Ezetimibe (Vytorin ®) |
| Rosuvastatin + Fenofibric Acid | Simvastatin + Niacin (Simcor ®) |
| (Centriad ®) | |

### Key Exclusion Criteria:

(a) Medical History:

Any surgical or medical condition that may interfere with absorption, distribution, metabolism, or excretion of niacin; history of extreme triglyceridemia (TG>1000 mg/dL) or pancreatitis from triglyceridemia, regardless of whether it is currently controlled; medical condition that would prohibit fasting (e.g. diagnosis of insulinoma or postabsorptive hypoglycemia);

Significant disinclination to dairy products (e.g., lactose intolerance, inviolable dietary restrictions)—all participants receive a test dose of the fat challenge during the screening visit, which consists of heavy cream and lactase enzyme. Many people with lactose intolerance successfully avert symptoms by correcting their lactase deficiency with lactase supplements. These people are required to tolerate the test dose given during screening;

Donation of whole blood within 8 weeks prior to the first experimental visit;

History of a non-skin malignancy within the previous 5 years;

Uncontrolled thyroid disease;

Any major active rheumatologic, pulmonary, or dermatologic disease or inflammatory condition;

Major surgery within the previous 6 weeks;

Subjects who have undergone any organ transplant;

History of drug abuse within the past 3 years, or regular alcohol use of greater than 14 drinks per 16 week;

Women who are breast-feeding;

Serious or unstable medical or psychological conditions that, in the opinion of the investigator, would compromise the subject's safety or successful participation in the study;

Known intolerance or contraindication to niacin (e.g. moderate to severe gout, severe peptic ulcer disease);

Subject-reported history of HIV and/or use of HIV medications.

(b) Physical and Laboratory:

Homocysteine <8 μmol/L or >25 μmol/L;

Triglycerides >500 mg/dL;

LDL >190 mg/dL;

Chronic renal insufficiency: serum creatinine >2.0 mg/dL or creatinine clearance <60 mLimin/1.73 m² by the MDRD Study equation;

Hypoalbuminemia, with a serum albumin of <2.5 mg/dL;

Hemoglobin <10 g/dL;

Women who are found to be pregnant by urine pregnancy test conducted at a study visit.

Drug Administration

Niacin is titrated to 3000 mg daily, initially as Slo-Niacin® from 500 mg to 2000 mg daily, and then switching to Niacor® 3000 mg daily. Table 5 summarizes the model titration strategy.

TABLE 5

| | | | |
|---|---|---|---|
| Week | Drug | Daily Dose | Schedule |

Niacin initiation phase with lower-flush
extended-release niacin (Slo-Niacin ®)

| | | | |
|---|---|---|---|
| 0 to 1 | ER Slo-Niacin ® | 500 mg | 500 mg nightly |
| 1 to 2 | ER Slo-Niacin ® | 1000 mg | 500 × 2 = 1000 mg nightly |
| 2 to 3 | ER Slo-Niacin ® | 1500 mg | 500 × 3 = 1500 mg nightly |
| 3 to 4 | ER Slo-Niacin ® | 2000 mg | 500 × 4 = 2000 mg nightly |

Niacin maintenance phase with cardioprotective
doses of immediate-release niacin (Niacor ®)

| | | | |
|---|---|---|---|
| >4 | IR Niacor ® | 3000 mg | 500 × 2 = 1000 mg thrice |
| | | | daily: breakfast, lunch, and |
| | | | supper |

In practice, the final dose and rate of titration to each subject is individualized, as needed to optimize tolerability. Individualized titration is important for niacin therapy because individuals accommodate to flushing at differing rates. The schedule above depicts the fastest dose and highest dose of titration, so that variations occur in the direction of slower titration or lower dose. During the niacin initiation phase, subjects are advised to take the aspirin 30 min prior to the nightly dose of ER Slo-Niacin®. During the maintenance phase with cardioprotective doses, subjects are advised to take the aspirin 30 min prior to the morning dose of IR Niacor. Dosing is not necessarily linked to daily events (night and meals).

After the fourth week, tolerability is assessed based on subjective symptom and safety laboratory testing. Subjects who tolerate 2 grams of Slo-Niacin® progress to the minimal cardioprotective dose by switching to immediate-release Niacor® as 1000 mg (500 mg tab×2) dosed three times a day with meals, for a total of 3 grams daily. The patient is evaluated 2 weeks after starting Niacor® to again evaluate tolerability. If a subject does not tolerate this dose, either by subjective symptoms or clinically-significant laboratory abnormalities, they are allowed to continue the previously-tolerated dose (Slo-Niacin® 2 grams nightly), and are again followed up a week later to assess tolerability.

### Example 3

### Visits 2, 3 and 7—Provocative Metabolic Challenge Studies

Three visits of the study involve prolonged visits during which subjects undergo provocative physiologic challenges to clarify the effects of the study treatments (Visits 2, 3, and 7). The first experimental visit is within 12 weeks of the screening visit. The procedures to be followed at each visit are briefly described below.

The subject is advised to avoid alcohol in general during the study. Subjects are asked to eat supper no later than 6 pm the night before presenting the following morning. Following supper the night before the metabolic challenge studies, subjects do not eat a conventional meal until completion of the visit, though they are encouraged to drink copious amounts of water and other non-caffeinated, non-carbonated, sugar-free drinks. Subjects do receive calories in the form of glucose during the clamp study, and as the fat shake during the oral fat tolerance test. Thus, they are relatively hypocaloric, but not acaloric following supper the night before.

An intravenous catheter (BD Intracath®) is placed in the antecubital vein for infusion during the clamp and blood is drawn during the oral fat tolerance test. Blood is collected from the IV to conduct baseline/safety laboratory work. Infusion of isotopically labeled glucose is started to measure endogenous glucose production. After two hours of the infusion, the glucose clamp study is started. Subjects provide a urine specimen that represents the pre-drug state. If the patient opts to undergo pharmacological liver function testing, the first indocyanine green study is conducted prior to the clamp and at two other times during the visit. The clearance of this marker is measured non-invasively using the optical skin probe attached to the index finger.

Euglycemic Hyperinsulinemic Clamp Study:

Blood is collected for assessment of basal parameters about 1.5 hours after the isotopically labeled glucose infusion commences. The clamp itself begins two hours after the infusion of isotopically labeled glucose starts. The isotopically labeled glucose infusion and hyperinsulinemic-euglycemic clamp are well-established procedures. The duration of the primed continuous insulin infusion is two hours, and plasma glucose is maintained at the basal level for 150 minutes, with an infusion of 20% dextrose. These continue for two hours, with additional blood collections toward the end of the clamp period. Blood is also collected during the hour immediately following clamp cessation to evaluate the return to baseline.

Oral Fat Tolerance Test:

The Oral Fat Tolerance Test differs during Visit 2, Visit 3 and Visit 8 in that the latter two include dosing of the chronic study medications. During Visit 3, subjects take MetanX with aspirin when the infusion is completed. During the followup visit (Visit 8), subjects take their first daily dose of niacin and MetanX with aspirin when the insulin infusion is completed (i.e., open-label Niacor® and blinded vitamins vs placebos). Apart from study drug administration and dispensing, the visits are identical. After the insulin infusion is completed, subjects wait for an hour before commencing the oral fat tolerance test. This delay also allows the niacin to be absorbed before the start of the test. Next, the subject drinks the oral fat load of heavy cream, which includes the amino acid methionine for the post-methionine load homocysteine challenge. The dietician adds lactose drops to the heavy cream, and if the subject has lactose intolerance, they may also take their preferred over-the-counter lactase enzymes. The Oral Fat Tolerance Test involves serial blood draws to evaluate pharmacodynamic effects of niacin on postprandial lipoproteins. Subjects rate flushing perception using the Niacin Tolerance Survey 1 hour after the start of the Oral Fat Tolerance Test. This allows one to assess distinguish flushing from niacin (Visit 8) from postprandial flushing (Visits 2 and 3). During the followup visit (Visit 8), their second daily dose of the study medications are administered 3 hours after taking the fat load. At the conclusion of the Oral Fat Tolerance Test, subjects eat a mixed meal prior to discharge. Apart from the fat load itself, subjects do not receive calories from meals until prior to discharge. Orthostatic blood pressure and pulse are measured to monitor for hypotension.

During the first metabolic study visit (Visit 2), blinded Metanx® (B6, B9, and B12) or respective placebos are dispensed to be taken chronically. In visits 4, 5 and 6, subjects are subjected to medication monitoring.

### Example 4

### Provocative Physiologic Challenges

### Carbohydrate Challenges:

Isotopically Labeled Glucose Methodology:

The measurement of endogenous (hepatic) glucose production and overall glucose disposal rates will be determined by tracer methodology using a primed-continuous infusion of isotopically labeled glucose. Tracer techniques may be safely applied to clinical studies to eliminate the obvious disadvantages and risks of catheterization methods. For example, when glucose tritiated in the hydrogen of the third carbon position is used, the tritium label is not metabolized into glycolytic intermediary compounds, but is eliminated as tritiated water. Therefore, when free water is removed from plasma samples by evaporation or lyophilization, the remaining counts are a measure of 3-3H-glucose specific activity and are used to calculate parameters of the metabolism of glucose. The glucose kinetics is calculated according to the steady-state equations.

Hyperinsulinemic Euglycemic Clamp Methodology:

The clamp study takes place in the morning after an overnight fast. Through an intravenous catheter isotopically labeled glucose is infused (at −120 min) to estimate glucose kinetics. Around 70 minutes after the start of the glucose infusion, a second intravenous catheter is inserted in a retrograde fashion in to the dorsal vein of the hand or wrist, and the hand is enclosed in a box heated to 68° to 72° C. in order to arterialize the venous blood. At −30 min four blood samples are drawn in EDTA every 10 minutes (−30 min, −20 min, −10 min, and −1 min) to assess basal plasma parameters, after which the hyperinsulinemic-euglycemic clamp procedure is initiated. Starting at minute 0, insulin and glucose are infused for 2 hours. Throughout the clamp study, the canulated hand remains in the heated chamber (68° to 72° C.). In addition to the blood samples at −30 min, −20 min, −10 min, and −1 min, blood is sampled at selected intervals between 90 and 150 minutes, with additional 0.5 mL samples every 5 minutes for glucose. At 120 minutes, the IV insulin is stopped and blood glucose is monitored for another half hour. Glucose infusion continues as necessary. The glucose infusion terminates when it is documented that the blood glucose levels are stable or hyperglycemic but not hypoglycemic.

### Postprandial Challenges:

Methionine Challenge:

The methionine challenge is a well-established test to quantify homocysteinemia, and simply involves administering L-methionine 0.1 g/kg body weight dissolved in orange juice. Homocysteine levels are measured prior to the methionine load and several hours after dosing. For practical purposes, a 6-hour post-methionine load reading is preferred. In addition to a 6-hour sample, intermediary time points are also recorded to better define the homocysteine curve following methionine loading.

Oral Fat Tolerance Test:

An oral fat challenge developed in prior studies is used to assess postprandial lipidemia. Oral fat is selected instead of intravenous fat loading, because a major antilipolytic hormone requires the intact chylomicrons from a meal as part of its signaling mechanism. A fat challenge is preferred over a glucose challenge or mixed challenge to study postprandial lipidemia, because it minimizes confounding by the anti-lipolytic hormone insulin (which is stimulated by a mixed meal with more carbohydrate). The fat load consists of a 40% (wt:vol) fat emulsion, with a polyunsaturated to saturated fat ratio of 0.1, and contains 0.001% (wtvol) cholesterol, and 3% (wt:vol) carbohydrates, with a total energy content of 3,700 kcal/L. The fat is mixed 18 hours prior to the visit and also includes 120,000 U aqueous vitamin A, dosed at 50 g per m² of body surface area.

The Mosteller formula is used to estimate body surface area:

$$B S A ( {m}^{2} ) \approx \sqrt{\frac{Height ( cm ) * Weight ( kg )}{3600}} \approx \sqrt{\frac{Height ( in ) * Weight ( lbs )}{3131}}$$

Subjects drink the fat load within twenty minutes, followed by 200 ml of water. Though subjects do not eat or drink conventional foods for the remainder of the day, they may consume water and non-caffeinated, calorie-free diet drinks. To limit temperature changes, drinks are served at room temperature. After completing the observation period, subjects are offered a meal prior to leaving. The fat challenge is analyzed primarily by plasma TG levels, but on an exploratory basis also separate chylomicrons and VLOL to measure TG, cholesterol, and retinyl palmitate levels in these subfractions. The latter is a marker to distinguish alimentary lipoproteins.

### Hepatic Function Testing:

Real-Time Evaluation and Management of Niacin-Induced Transaminasemia During the Trial:

True niacin-induced hepatotoxicity has proven too rare to be detected in controlled trials. However, transaminasemia is occasionally seen in trials, and is viewed as a surrogate, albeit weak, indicator of adverse effects on the liver if left unattended. A subclinical increment in transaminases is not associated with clinical adverse events unless accompanied by true changes in liver function (e.g., elevated bilirubin or coagulation factors). Given the reversibility of niacin-induced transaminasemia, markers of hepatic status offer a reasonable technique to infer adverse effects on liver in a systematic way. This protocol is designed to minimize the potential for risk from hepatotoxicity. For example, niacin formulations used in the study (Niacor® and Slo-Niacin®) are well studied, having been subjected to extensive clinical and research testing.

Limitation of Traditional Biochemical Surrogates of Hepatic Function:

A persistent difficulty of studying hepatic outcomes is the lack of sensitive tests of liver function. The transaminases give clues about adverse hepatic effects that might be severe enough to accelerate hepatocellular turnover, and represent increased senescence of hepatocytes or outright toxicity. This assumes that niacin does not increase the synthesis of the transaminases, as at least one other non-statin does. True tests of hepatic synthetic function include bilirubin levels and tests of the coagulation system. Unfortunately, both transaminases and clinical tests of synthetic function are insensitive. Reliance on these tests may have led to a considerable underestimation of the adverse hepatic effects of niacin.

Hepatic Function Testing by Indocyanine Green Clearance:

Hepatic function is assessed using a standardized injection of indocyanine green. Briefly, 0.5 mg/kg body weight is injected into the peripheral IV line as a bolus. Due to the extremely short half-life, the dye is typically present for 15 to 20 minutes, so that the clearance may be defined rapidly at the bedside. The clearance of indocyanine green is well established, and clearance parameters have been described by compartmental modeling (Tichy et al., 2009, Physiol. Res. 58(2):287-92). In practice, the parameters are calculated by the LiMON measurement device (Pulsion Medical Systems, Munich, Germany). This device uses low-energy, visible light to measure the intensity of the dye in the finger tip. This is similar to pulse oximetry, which shines low-energy red light onto the fingertip to estimate oxygen saturation.

### Example 5

### Laboratory Evaluations

### Clinical Laboratory Tests Evaluated in Real Time:

The Clinical Chemistry Panel consists of an extended electrolyte panel (sodium, potassium, chloride, bicarbonate, phosphorous, calcium, and magnesium), urate, BUN, creatinine, and glucose. The Clinical Carbohydrate Panel consists of glucose, fructosamine, and hemoglobin A1C. The Hepatic Panel consists of ALT, AST, bilirubin, albumin, Alk Phos, gGGT, and coagulation panel. The Methylation Panel consists of homocysteine and vitamins B6, B9 (folate), and B12. The Complete Blood Count (CBC) consists of hemoglobin, hemotacrit, WBC count, platelet count, MCV, MCH, MCHC, RDW. The CBC is also used during each experimental visit as a pharmacodynamics outcome. As a pharmacodynamic outcome, the WBC count and the automated leukocyte differential are used to determine the absolute neutrophil count (ANC).

### Laboratory Tests on Stored Plasma Samples:

The pharmacodynamic outcomes based on laboratory analyses are conducted on frozen plasma samples after a subject has completed a given experiment, so that the tests may be run in batches. Since this is a progressive chronic therapy study, a given subject's samples are preferably analyzed in the same run or session.

Plasma Processing and Storage:

Blood is collected into EDTA tubes that are pre-chilled on ice, and are again placed on ice immediately after collection. Within ½ hour of collection, plasma is separated using a centrifuge chilled to 4° C. for 15 minutes at 3,000 rpm. The plasma is collected into cryovials for long-term storage in a −70° C. freezer until laboratory analysis. Because FFA and insulin are especially sensitive to multiple freeze/thaw cycles, they are analyzed on the first thaw. Whenever possible, clinical chemistry analyzers with multiplexing capabilities are used so conserve plasma and limit multiple freeze/thaw cycles.

Evaluation of the Systemic Inflammatory Response to Niacin:

Cytokines, hsCRP, and ANC are evaluated as the primary outcomes for the systemic inflammatory response to niacin. The effect of niacin on leukocyte mRNA expression is also evaluated. Both peripheral leukocytes and the leukocytic infiltrate of dermal tissue are potential mediators of the IL-6 surge. Since leukocytes express the GPR109A receptor, niacin could have direct effects on leukocyte activation. Whole blood is collected using PAX tubes to isolate whole blood RNA prior to and following niacin administration. mRNA is extracted using the PAXgene kit (Qiagen, Inc) to analyze changes in IL-6 mRNA by reverse transcription followed by competitive polymerase chain reaction amplification (RT-PCR). Based on the interim analysis of the first several subjects, if a meaningful increase in IL-6 mRNA is observed, appropriate separation techniques may be used to further isolate candidate leukocytes. For example, Ficoll separation of the mononuclear cell fraction may be used.

### Laboratory Tests on Stored Urine Samples:

Urinary eicosanoids including thromboxane are measured prior to and following niacin exposure. Because eicosanoids are short-lived, surrogates are used to infer their activity. A urinary metabolite of thromboxane is measured by mass spectrometry. Following the addition of a deuterated isoprostane internal standard, urine samples are extracted from the aqueous matrix by solid phase extraction techniques, derivatized, and analyzed by LC/MS/MS using multiple reaction monitoring (MRM) techniques. Quantification is accomplished by taking the ratio of the area under the peak of the ion representing the endogenous compound to that of the internal standard.

### Laboratory Tests on Stored Adipose Samples:

Biopsy Technique:

Gluteal adipose biopsies are conducted prior to and following niacin dosing to clarify the effect of the drug on subcutaneous adipose. A field on the buttock is sterilized, with the patient in the prone position, and a small area is injected with 1 or 2% lidocaine without epinephrine. Then a small incision is made in the skin to allow the introduction of a fenestrated blunt-ended liposuction catheter attached to a 60 cc syringe. Approximately 10 cc are injected into the subcutaneous adipose to loosen the tissue, and the tissue is agitated under manual suction to remove 1-2 g of adipose tissue. The edges of the wound are brought together and then a 4 mm punch biopsy of skin and underlying adipose is conducted. The skin is then cleaned, and the biopsy site sutured closed, and bandaged. The subject is then repositioned to sit with their weight on the site to aid hemostasis.

Adipose from the liposuction biopsy is dissected in a cold field to remove obvious blood clots, and wash it with chilled normal saline. The adipose is partitioned into cryovials and snap frozen, and then stored in a −70° freezer.

Deep Subcutaneous Adipose mRNA Expression:

Deep subcutaneous adipose mRNA is extracted using the RNeasy total RNA kit (Qiagen), providing approximately 1-4 μg RNA per 100 mg tissue. mRNA concentrations for candidate genes are determined by RT-PCR. Candidate genes include BHMT, NNMT, PEMT, IL-6, and adiponectin, but are not limited to this list. As an example of the approach, IL-6 concentrations are normalized to beta-actin as a housekeeping gene, and the relative change in IL-6 mRNA before and after dosing is compared under the treatment combinations. If there is sufficient sample, mass of key compounds (e.g. SAH and SAM) is also measured.

### Example 6

### Gross Evaluation of the Dermal Response to Niacin (e.g. Flushing)

Because little is known of the change in objective measures of flushing over time, acutely-provoked flushing prior to and during chronic treatment with niacin is measured.

### Topical Niacin Flushing Capacity Test:

Because some individuals lack the capacity to flush from niacin, a Topical Niacin Flushing Capacity Test is conducted by applying aqueous methyl nicotinic acid (Sigma, Inc) to a small portion of the forearm during the screening visit. Directly applying nicotinic acid to the skin is a well-established technique in investigative dermatology and is well tolerated.

Using the same technique, the response to the topical application of methyl nicotinic acid to the skin of the forearm is assessed, using standardized concentrations of 0.1 M, 0.01M, and 0.001 M. First, a hairless area on the volar forearm that is free of tattoos and has relatively few endogenous pigmentary discontinuities (e.g. freckles, hyperdensities, hypodensities) is selected. To identify the skin patch for each test dose, a paper card is affix to the forearm, wherein the card has pre-cut holes that expose skin at appropriate intervals. Next, 50 microliters of each concentration are applied to filter paper, which is then applied to the skin for 90 seconds. Erythema is assessed with the Berger 7-point visual scale at baseline and every 5 minutes with a final assessment at 15 minutes. Optical techniques described elsewhere (colorimetry, laser Doppler flowmetry, or both) are also used. When using colorimeter to evaluate dermal flushing of the forearm, it is likely that dark or thick arm hair will negatively bias readings on the L* axis, and red arm hair will positively bias the results toward the a* axis of the reflectance values. This bias is minimized by wet shaving a small portion of the volar forearm.

The Berger scale is as follows: 1—No skin reaction; 2—Confluence of red spots in less the 50% of total patch area; 3—Slight redness/confluence of red spots in more than 50% of patch area; 4—Moderate redness/homogenous erythema of who patch area; 5—Spreading redness and/or visible edema; 6—Visible edema encompassing whole patch area; 7—Visible edema that starts to spread out/edema bigger than the original patch area.

### Objective Measures of Physical Stigmata of Flushing:

The physical stigmata of niacin-induced flushing is objectively measured. Specifically, hyperemia is measured by laser Doppler flowmetry, warmth by colorimetry, and erythema by colorimetry. These techniques provide complementary information. Specifically, each has limitations that are not shared by one or more of the others, justifying the use of more than one technique. Flowmetry was selected as the primary index of flushing, along with the other two supporting modalities.

Measuring Hyperperfusion with Laser Doppler Flowmetry:

Laser Doppler flowmetry (Moor Instruments Ltd, Devon, UK) is used as an objective gauge of cutaneous hyperperfusion in the face in response to niacin specifically, at the malar eminence. This method uses external skin probes that measure the perfusion of the microcirculation in the target area. Using the Doppler effect, this machine measures the frequency of light scattered by moving erythrocytes. The frequency signal is proportional to erythrocyte concentration, which is mathematically related to the velocity and flow of the blood. For example, lower frequency represents lower velocity, while higher frequency represents higher velocity. The flowmeter samples blood flow at a rate of 40 Hz. For practical reasons, this is reduced to 1 Hz for analysis because there may not be an advantage to such a high sampling rate, but the higher rate increases computation time. At first glance, this would seem to be an ideal way to objectify flushing. However, laser Doppler flowmetry is limited by the occurrence of edema that follows niacin exposure. The pressure of the edema may constrict the blood vessels, thereby attenuating the superficial vasodilatory response and perhaps reducing the accuracy of this assessment. This is another reason to use multiple modalities to quantify flushing.

Measuring Warmth with Thermometry:

Warmth is measured by thermometry, to calculate the change in malar thermal circulation index (ΔMTCI), developed by Wilkin. In the equation that follows, Tmb is the baseline malar temperature, Tab and Tcb are the ambient and core temperatures at baseline, Tmp is the maximum malar temperature, and Tap and Tcp are the ambient and core temperatures at the time of the peak malar temperature. Temperature is measured using flexible thermister wires connected to a data logger that records temperatures every 10 seconds (Minilogger, Respironics, Inc, Bend, Oreg.).

$$Δ M T C I = \frac{Tmp - Tap}{Tcp - Tmp} / \frac{Tmb - Tab}{Tc b - Tmb}$$

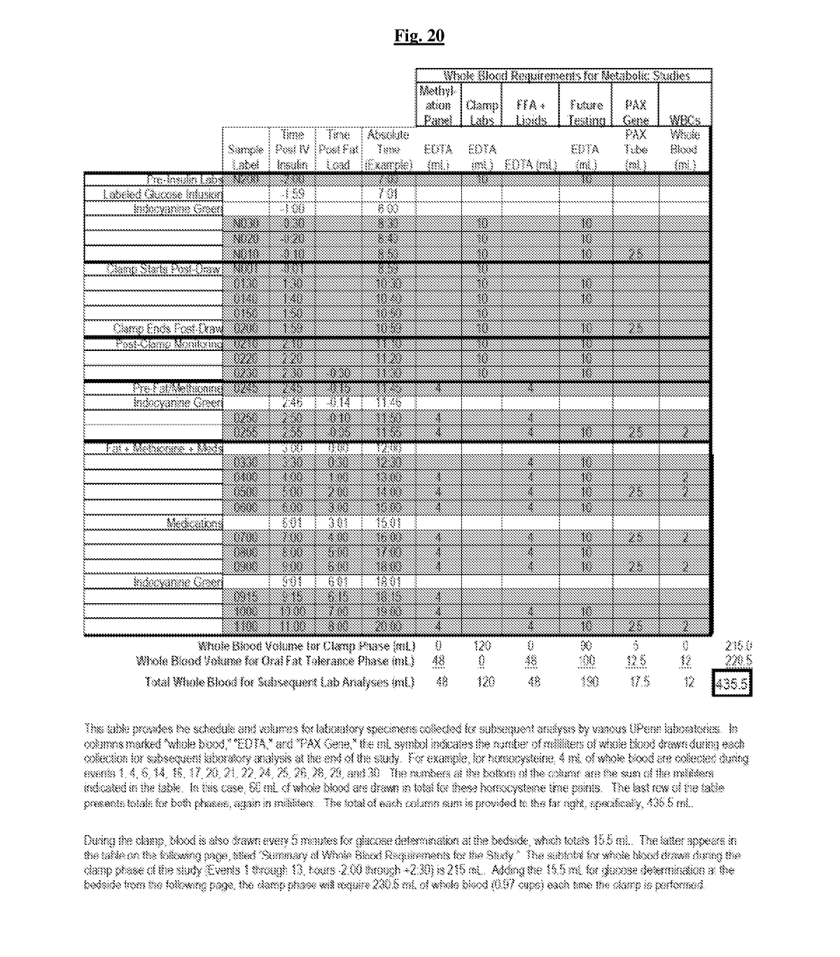

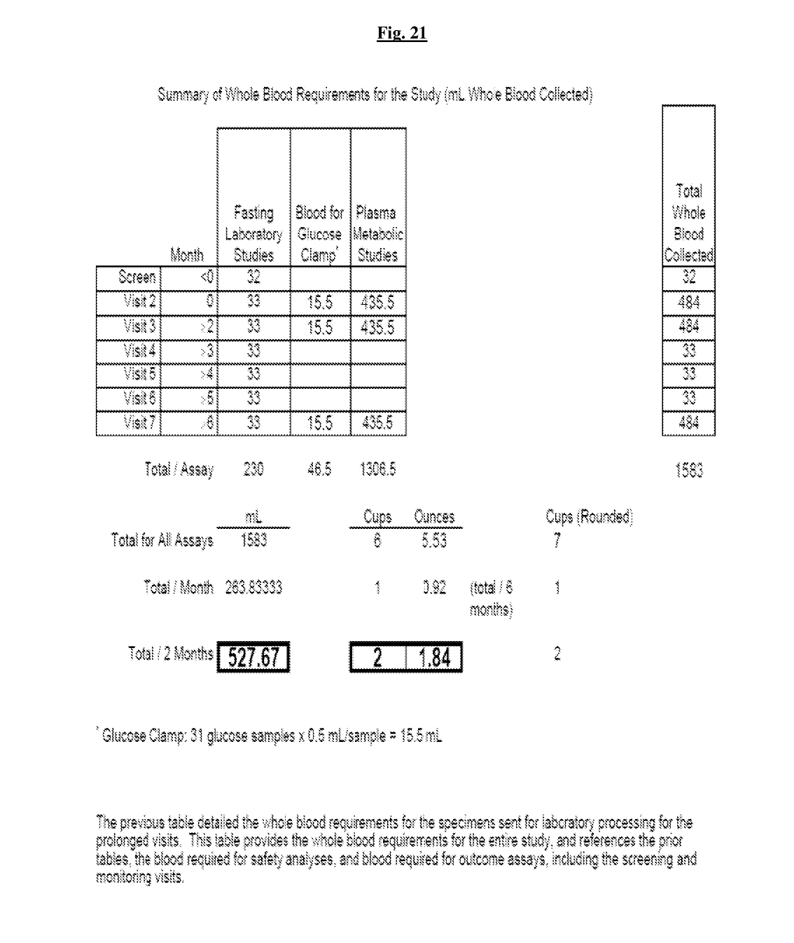

Valid measurements of core temperature and continuous data recording are advisable. Unfortunately, the traditional surrogate markers of core temperature may be biased in a variable and unpredictable manner by interventions such as niacin that selectively increase surface temperature in the upper body. For example, tympanic membrane temperature may be likely biased because flushing from niacin affects the pinna and probably the ear canal. Likewise, axillary temperature may be biased by increased dermal blood flow. Sublingual temperature may also be biased by the increased temperature of the head and neck. More reliable sources for core temperature are esophageal temperature using a thin flexible thermister inserted in the nostril, or rectal temperature, also using a thin flexible thermister (FIG. 20). Skin temperature is measured using a set of dermal thermisters attached to the malar prominence, and ambient temperature with a thermister attached to head of the subject's bed.

Measuring Erythema with Colorimetry:

Colorimetry is used to objectively measure niacin-induced flushing. Medical colorimeters express color in numerical values, and are useful to observe color differences within and between subjects. Color perception by the naked eye is affected by illumination, surrounding environment color, and observation angle. By limiting these biases, the colorimeter assures uniformity and reproducible results. Colorimeters use an optical method that imitates human color perception, and evaluates responses to light stimuli, which are measured by standardized color systems. Colorimetry is well-suited to measure cutaneous flushing from niacin, because one of the physical stigmata is dermal erythema. Accordingly, a handheld colorimeter is used to quantify erythema at baseline and for several hours after niacin dosing. Specifically, erythema is measured by isolating the red vector from colorimetric measurements. The erythema outcome is based on the change in the magnitude of the red vector from the pre-dose measurement. Colorimetry may have an advantage of thermometry, because core temperature is not constant during the observation interval because of normal diurnal variation. Moreover, changes are anticipated in core temperature from niacin. Thus, thermometry may lose some strength of association with flushing symptoms at later hours following a dose of niacin, because the skin remains hyperemic, but the blood is of lower temperature. Colorimetry would retain its strength of association with flushing in this time period. A disadvantage of colorimetry is that melanin absorbs the same wavelength of green light as hemoglobin. Thus, the absolute values of erythema vary slightly across individuals based on melanin concentration. It is also possible that the magnitude of the increment in erythema may vary slightly because of this interaction. Thus, the y-axis on a pharmacodynamic curve may be difficult to compare across individuals. However, the time axis is not affected by this interaction, so that colorimetric data allow us to evaluate the timing of flushing, symptomatic and laboratory events Importantly, event analysis in the later hours of the study would not be distorted by a change in the reference variable, which is a potential limitation of thermometry.

When available, as a corroboratory test, erythema is assessed directly with a clinical colorimeter using a fiber optic cable attached to the malar prominence (CyberDerm, Inc, PA). This is analyzed by change in the red/green vector (a*), as defined by the Commission Internationale de l'Eciairage (CIE) L*a*b* color space.

### Quantifying Perception of Flushing Symptoms:

The physical measurements of flushing stigmata do not fully inform one as to niacin tolerability because some of the more irritating aspects of niacin defy physical measurement (e.g., itching, burning). Therefore, flushing perception is also measured using semi-quantitative surveys. Subjects rate flushing perception at screening using the modified Global Flushing Severity Score (GFSS) and the Niacin Tolerance Survey one hour after they receive the test dose of niacin. During experimental visits, subjects rate flushing perception hourly using the FAST tool and 2 hours post-niacin using the Niacin Tolerance Survey.

The disclosures of each and every patent, patent application, and publication cited herein are hereby incorporated herein by reference in their entirety.

While the invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed:

1. A method of reducing or preventing the increase of triglyceride levels in a mammal in need thereof, the method comprising administering to the mammal a therapeutically effective amount of a pharmaceutically acceptable composition comprising a lipid-managing medication, wherein the mammal is further administered a methylation enhancing supplement,
wherein the lipid-managing medication comprises niacin, a fibrate, or a prodrug, analogue or metabolite thereof,
thereby reducing or preventing the increase of triglyceride levels in the mammal.

2. The method of claim 1, wherein the methylation enhancing supplement comprises betaine, serine, methionine, s-adenosyl methionine, choline, phosphatidylcholine, creatine, cysteamine, cysteine, N-acetylcysteine, alpha lipoic acid, melatonin, silymarin, vitamin B5, pantethine, whey protein, vitamin B6, vitamin B9, vitamin B12, any salts thereof, or any combinations thereof,
wherein the methylation enhancing supplement is not silymarin if the lipid-managing medication comprises niacin or a prodrug, analogue or metabolite thereof.

3. The method of claim 1, wherein the lipid-managing medication and the methylation enhancing supplement are separately administered to the mammal.

4. The method of claim 1, wherein the lipid-managing medication and the methylation enhancing supplement are concomitantly administered to the mammal.

5. The method of claim 4, wherein the lipid-managing medication and the methylation enhancing supplement are co-formulated for administration to the mammal.

6. The method of claim 1, wherein the lipid-managing medication is in a controlled release formulation.

7. The method of claim 6, wherein the lipid-managing medication is in an intermediate-release or extended-release formulation.

8. The method of claim 1, wherein the mammal develops less insulin resistance than when the mammal is administered a similar dose of lipid-managing medication and is not administered the methylation enhancing supplement.

9. The method of claim 1, wherein the mammal develops less tachyphylaxis than when the mammal is administered a similar dose of lipid-managing medication and is not administered the methylation enhancing supplement.

10. The method of claim 1, wherein the mammal develops less hepatotoxicity than when the mammal is administered a similar dose of lipid-managing medication and is not administered the methylation enhancing supplement.

11. The method of claim 1, wherein the mammal is administered from about 2 grams to about 6 grams of the lipid-managing medication daily.

12. The method of claim 2, wherein the mammal is administered at least one daily dose selected from the group consisting of about 50 mg of vitamin B6, about 5 mg of vitamin B9, about 4 μg of vitamin B12, and about 2 grams to about 6 grams of betaine.

13. The method of claim 1, wherein the mammal is a human.

14. A method of increasing or preventing the decrease of HDL levels in a mammal in need thereof, the method comprising administering to the mammal a therapeutically effective amount of a pharmaceutically acceptable composition comprising a lipid-managing medication, wherein the mammal is further administered a methylation enhancing supplement
wherein the lipid-managing medication comprises niacin, a fibrate, or a prodrug, analogue or metabolite thereof;
thereby increasing or preventing the decrease of HDL levels in the mammal.

15. The method of claim 14, wherein the supplement comprises betaine, serine, methionine, s-adenosyl methionine, choline, phosphatidylcholine, creatine, cysteamine, cysteine, N-acetylcysteine, alpha lipoic acid, melatonin, vitamin B5, pantethine, silymarin, whey protein, vitamin B6, vitamin B9, vitamin B12, any salts thereof, or any combinations thereof,
wherein the supplement is not silymarin if the lipid-managing medication comprises niacin or a prodrug, analogue or metabolite thereof.

16. The method of claim 14, wherein the lipid-managing medication and the methylation enhancing supplement are separately administered to the mammal.

17. The method of claim 14, wherein the lipid-managing medication and the methylation enhancing supplement are concomitantly administered to the mammal.

18. The method of claim 17, wherein the lipid-managing medication and the methylation enhancing supplement are co-formulated for administration to the mammal.

19. The method of claim 14, wherein the lipid-managing medication is in a controlled release formulation.

20. The method of claim 19, wherein the lipid-managing medication is in an intermediate-release or extended-release formulation.

21. The method of claim 14, wherein the mammal develops less insulin resistance than when the mammal is administered a similar dose of lipid-managing medication and is not administered the methylation enhancing supplement.

22. The method of claim 14, wherein the mammal develops less tachyphylaxis than when the mammal is administered a similar dose of lipid-managing medication and is not administered the methylation enhancing supplement.

23. The method of claim 14, wherein the mammal develops less hepatotoxicity than when the mammal is administered a similar dose of lipid-managing medication and is not administered the methylation enhancing supplement.

24. The method of claim 14, wherein the mammal is administered from about 2 grams to about 6 grams of the lipid-managing medication daily.

25. The method of claim 15, wherein the mammal is administered at least one daily dose selected from the group consisting of about 50 mg of vitamin B6, about 5 mg of vitamin B9, about 4 μg of vitamin B12, and about 2 grams to about 6 grams of betaine.

26. The method of claim 14, wherein the mammal is a human.

27. A pharmaceutical composition comprising a lipid-managing medication and a methylation enhancing supplement, wherein the lipid-managing medication comprises niacin or a fibrate, or a prodrug, analogue or metabolite thereof.

28. The composition of claim 27, wherein the methylation enhancing supplement comprises betaine, serine, methionine, s-adenosyl methionine, choline, phosphatidylcholine, creatine, cysteamine, cysteine, N-acetylcysteine, silymarin, alpha lipoic acid, melatonin, vitamin B5, pantethine, silymarin, whey protein, vitamin B6, vitamin B9, vitamin B12, a salt thereof, or any combinations thereof,
wherein the supplement is not silymarin if the lipid-managing medication comprises niacin or a prodrug, analogue or metabolite thereof.

29. The composition of claim 28, wherein the lipid-managing medication and the methylation enhancing supplement are co-formulated for administration to the mammal.

30. The composition of claim 27, wherein the lipid-managing medication is in a controlled release formulation.

31. The composition of claim 30, wherein the lipid-managing medication is in an intermediate-release or extended-release formulation.

32. The composition of claim 27, comprising about 2 grams to about 6 grams of the lipid-managing medication.

33. The composition of claim 27, comprising at least one amount selected from the group consisting of about 50 mg of vitamin B6, about 5 mg of vitamin B9, about 4 μg of vitamin B12, and about 2 grams to about 6 grams of betaine.

34. A method of reducing or preventing the increase of triglyceride levels in a mammal in need thereof, the method comprising administering to the mammal a therapeutically effective amount of a pharmaceutically acceptable composition comprising a methylation enhancing supplement, thereby reducing or preventing the increase of triglyceride levels in the mammal.

35. The method of claim 34, wherein the methylation enhancing supplement comprises betaine, serine, methionine, s-adenosyl methionine, choline, phosphatidylcholine, creatine, cysteamine, cysteine, N-acetylcysteine, alpha lipoic acid, melatonin, vitamin B5, pantethine, silymarin, whey protein, vitamin B6, vitamin B9, vitamin B12, a salt thereof, or any combinations thereof.

36. The method of claim 34, wherein the methylation enhancing supplement is in a controlled release formulation.

37. The method of claim 36, wherein the methylation enhancing supplement is in an intermediate-release or extended-release formulation.

38. The method of claim 34, wherein the mammal is a human.

39. A method of increasing or preventing the decrease of HDL levels in a mammal in need thereof, the method comprising administering to the mammal a therapeutically effective amount of a pharmaceutically acceptable composition comprising a methylation enhancing supplement, thereby increasing or preventing the decrease of HDL levels in the mammal.

40. The method of claim 39, wherein the methylation enhancing supplement comprises betaine, serine, methionine, s-adenosyl methionine, choline, phosphatidylcholine, creatine, cysteamine, cysteine, N-acetylcysteine, alpha lipoic acid, melatonin, vitamin B5, silymarin, pantethine, whey protein, vitamin B6, vitamin B9, vitamin B12, a salt thereof, or any combinations thereof.

41. The method of claim 39, wherein the methylation enhancing supplement is in a controlled release formulation.

42. The method of claim 41, wherein the methylation enhancing supplement is in an intermediate-release or extended-release formulation.

43. The method of claim 39, wherein the mammal is a human.

44. A method of treating or preventing insulin resistance in a mammal in need thereof, the method comprising administering to the mammal a therapeutically effective amount of a pharmaceutically acceptable composition comprising a methylation enhancing supplement, thereby treating or preventing insulin resistance in the mammal.

45. The method of claim 44, wherein the methylation enhancing supplement comprises betaine, serine, methionine, s-adenosyl methionine, choline, phosphatidylcholine, creatine, cysteamine, cysteine, N-acetylcysteine, alpha lipoic acid, melatonin, vitamin B5, silymarin, pantethine, whey protein, vitamin B6, vitamin B9, vitamin B12, any salts thereof, or any combinations thereof.

46. The method of claim 44, wherein the methylation enhancing supplement is in a controlled release formulation.

47. The method of claim 46, wherein the methylation enhancing supplement is in an intermediate-release or extended-release formulation.

48. The method of claim 44, wherein the mammal is a human.
